(19) United States
(12) Patent Application Publication
(10) Pub. No.: US 2024/0386991 A1
(43) Pub. Date: Nov. 21, 2024

(54) SYSTEMS AND METHODS FOR ELECTROSTATIC LANDSCAPE OF MHC-PEPTIDE BINDING REVEALED USING INCEPTION NETWORKS

(71) Applicants: Eric Wilson, Tempe, AZ (US); Abhishek Singharoy, Chandler, AZ (US); Karen Anderson, Scottsdale, AZ (US); John Cava, Tempe, AZ (US)

(72) Inventors: Eric Wilson, Tempe, AZ (US); Abhishek Singharoy, Chandler, AZ (US); Karen Anderson, Scottsdale, AZ (US); John Cava, Tempe, AZ (US)

(73) Assignee: Arizona Board of Regents on Behalf of Arizona State University, Tempe, AZ (US)

(21) Appl. No.: 18/601,626

(22) Filed: Mar. 11, 2024

Related U.S. Application Data
(60) Provisional application No. 63/451,533, filed on Mar. 10, 2023.
(60) Provisional application No. 63/526,941, filed on Jul. 14, 2023.

(51) Int. Cl. G16B 15/30; G06N 3/0464; G16B 15/20; G16B 40/20

(52) U.S. Cl. CPC G16B 15/30; G06N 3/0464; G16B 15/20; G16B 40/20

(57) ABSTRACT

Predictive modeling of peptide binding motifs of major histocompatibility complex class I (MHC-I) is employed through the application of a predictive machine learning (ML) model. The predictive ML model can utilize structural and biophysical data obtained by modeling the physical structure of the binding pocket of the MHC-I and generating an electrostatic potential distribution of the binding pocket structural model. By utilizing the structural an biophysical data of the binding pocket of the MHC-I, the predictive ML model predicts the amino acid sequence of the peptide binding motif of the MHC-I.

### CROSS REFERENCE TO RELATED APPLICATIONS

The present non-provisional patent application claims priority to U.S. provisional patent application Ser. No. 63/451,533 filed on Mar. 10, 2023, and further claims priority to U.S. provisional patent application Ser. No. 63/526,941 filed on Jul. 14, 2023, the entirety of each of which is hereby incorporated by reference.

### FIELD

The present disclosure generally relates to computational methods; and in particular to systems and computational methods for prediction of MHC-I binding motifs using biophysical measurements of the MHC-I binding pocket, among other aspects described herein.

### BACKGROUND

Accurate modeling of macromolecular recognition and protein-protein complementarity represents one of the cornerstones of biophysical sciences. However, such models are often hindered by the combinatorial complexity of potential interactions at the molecular interfaces. Exemplary of this problem is peptide presentation by the major histocompatibility complex class I (MHC-I) molecules, a principal component of immune recognition. Recent advances in adoptive T cell therapies and vaccine development have highlighted the need for the accurate predictions of MHC-I binding. However, the extremely polymorphic nature of the MHC molecules makes any universal prediction of the peptide ligands difficult.

It is with these observations in mind, among others, that various aspects of the present disclosure were conceived and developed.

### BRIEF DESCRIPTION OF THE DRAWINGS

The present patent or application file contains at least one drawing executed in color. Copies of this patent or patent application publication with color drawing(s) will be provided by the Office upon request and payment of the necessary fee.

FIGS. 1A-1E are illustrations of a deep convolutional neural network, HLA-Inception, capable of predicting MHC-I peptide binding motif using data derived from the structure of the MHC-I binding pocket.

FIGS. 2A-2F are various graphs illustrating that electrostatic potential configuration space better captures MHC-I binding motif variation.

FIGS. 3A-3H are illustrations and graphs of exploration of predicted binging motifs.

FIGS. 4A-4F are graphs showing pan-allele peptide prediction with HLA-Inception.

FIGS. 5A-5C illustrate a schematic of the overall process of data generation and training of the HLA-Inception model described herein; and the generation of training data for the HLA-Inception training.

FIGS. 6A-6E are graphs illustrating protein modeling being robust to multiple point mutations.

FIG. 7 is a graph illustrating elector static cluster selection.

FIGS. 8A-8T are graphs illustrating hyperparameters tuning for MHC-I anchor positions.

FIGS. 9A-9C are graphs illustrating supertype network analysis.

FIGS. 10A-10L are a series of graphical representations showing HLA-A supertypes described herein.

FIGS. 11A-11L are a series of graphical representations showing HLA-B supertypes described herein.

FIGS. 12A-12L are a series of graphical representations showing HLA-C supertypes described herein.

FIGS. 13A-13C are graphs associated with concepts described herein.

FIGS. 14A-14J are graphs associated with concepts described herein.

FIG. 15 is a graph illustrating Leave One Cluster out analysis and computation of Matthew's correlation coefficient (MCC) calculated with respect to peptide prediction using the HLA-Inception model.

FIGS. 16A-16D illustrate peptide prediction.

Corresponding reference characters indicate corresponding elements among the view of the drawings. The headings used in the figures do not limit the scope of the claims.

### DETAILED DESCRIPTION

The present disclosure relates to examples of a structure-based deep learning framework for major histocompatibility complex class I (MHC-I) binding motif prediction. Examples of the present inventive concept can be implemented as a computer-implemented method, system, device, and/or machine-readable instructions that configure a processor to perform functions described herein.

In one example, the inventive concept is a method for predicting an amino acid sequence of a peptide binding motif of an MHC-I. The method can include modeling the binding pocket of the MHC-I. An electrostatic potential distribution of the model of the binding pocket of the MHC-I can be generated and input into a machine learning model. The machine learning model is executed using the electrostatic potential distribution and predicts the amino acid sequence of the peptide binding motif of the MHC-I based on the electrostatic potential distribution.

In another example, the inventive concept is a computer implemented system for prediction of MHC-I binding motifs. The computer implemented system includes a processor in operable communication a memory which contains instructions executable by the processor to predict the amino acid sequence of the MHC-I binding motif. The processor may predict the amino acid sequence of the MHC-I binding motif by accessing biophysical measurements of an MHC-I binding pocket, generating an electrostatic potential distribution based on the biophysical measurements, and executing a machine learning model. In some examples, the biophysical measurements can include structural data associated with the MHC-I binding pocket.

In yet another example, the inventive concept is a method for predicting an amino acid sequence of a binding peptide to an MHC-I. The method can include modeling the binding pocket of the MHC-I. An electrostatic potential distribution of the model of the binding pocket of the MHC-I can be generated and input into a machine learning model. The machine learning model is executed using the electrostatic potential distribution and predicts the amino acid sequence of the peptide binding to the MHC-I based on the electrostatic potential distribution.

### Introduction

Major histocompatibility complex I or MHC-I protein plays an integral role in permitting the immune surveillance of host cells and plays an integral role in successful viral clearance and tumor rejection. The MHC-I protein also denoted as human leukocyte antigen (HLA), drives molecular recognition by presenting endogenous peptide fragments on the cell surface for interaction with T cell receptors from the CD8⁺ T cells. Such processing and presentation pathways have the remarkable ability to present peptides from virtually any expressed cytosolic protein to the circulating T cells. Therefore, MHC-I proteins are an ideal system to study molecular recognition as it accommodates thousands to millions of potential protein-peptide interactions and presents an opportunity to bridge these details to phenotypic outcomes and biomedical consequences.

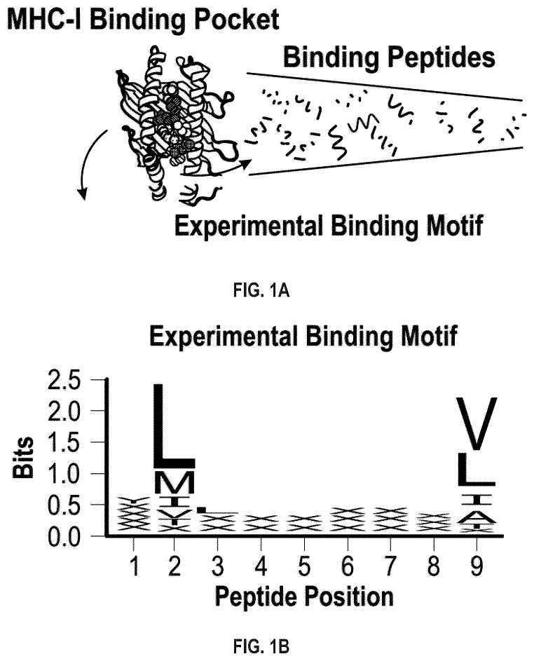

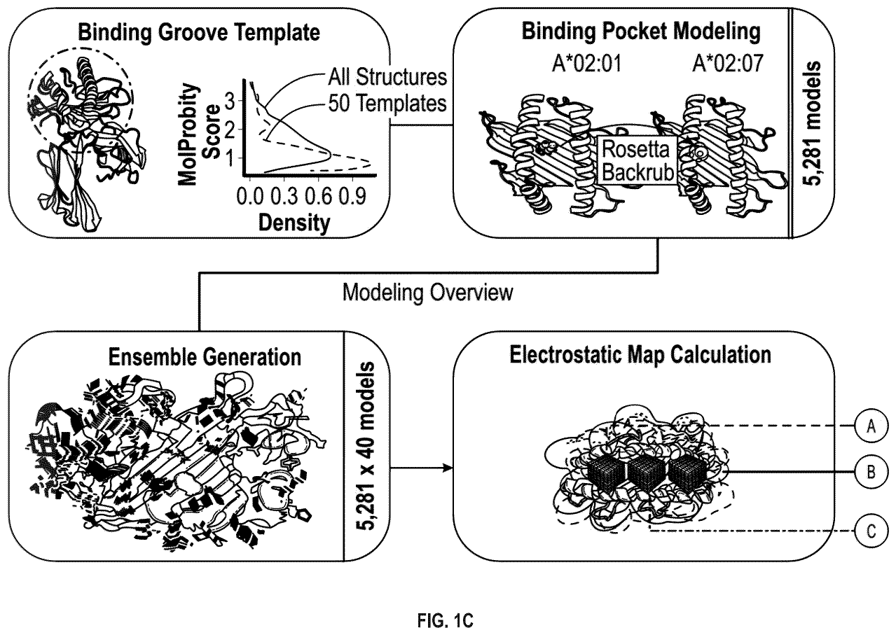

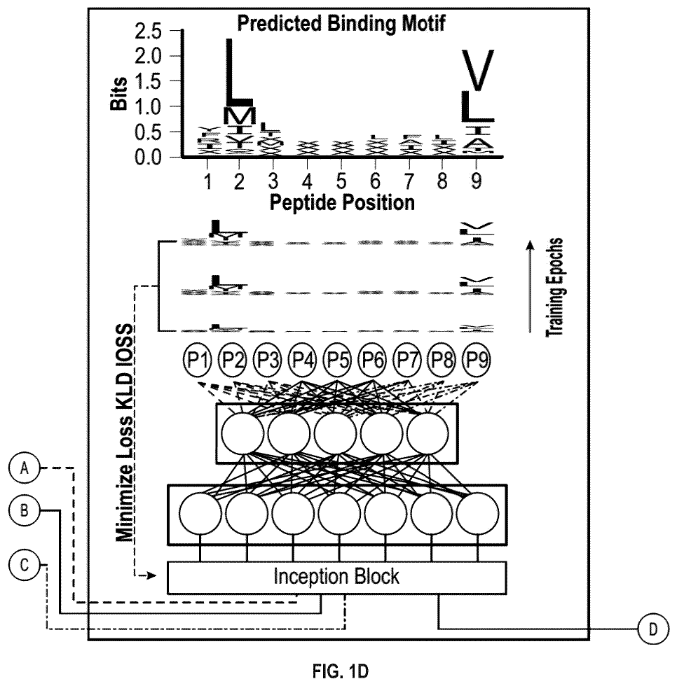

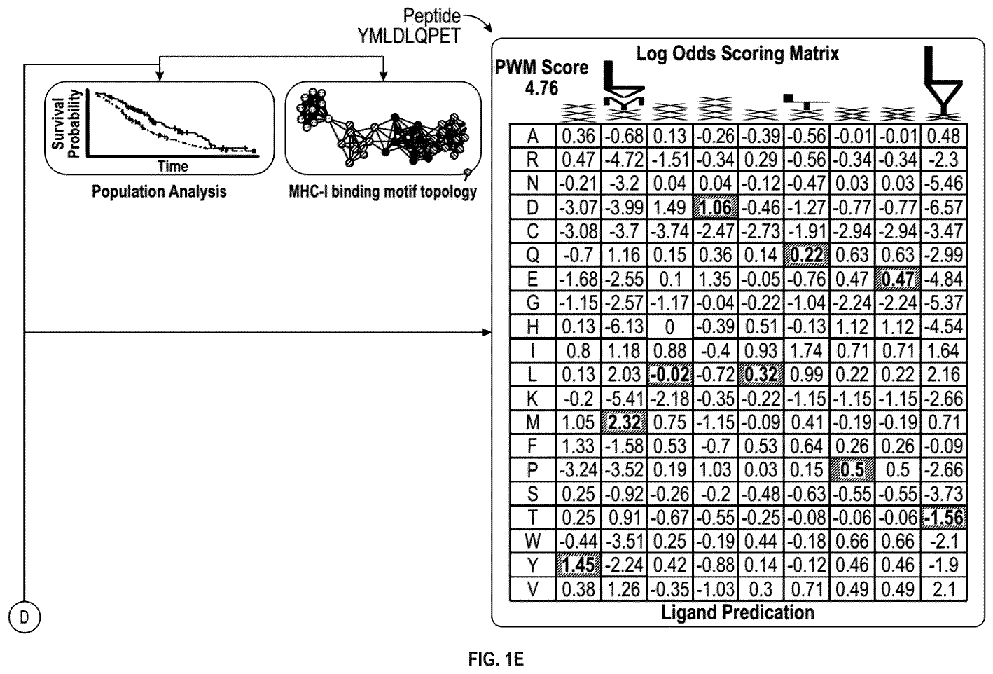

Referring to FIGS. 1A-1D, an example MHC-I complex ribbon structure depicts the MHC-I binding pocket capable of binding one or more peptides according to the graphically represented MHC-I binding motif. The MHC-I binding motif prediction ML model disclosed herein utilizes molecular models and the associated binding pocket electrostatic potentials of the MHC-I proteins. The construction of the molecular models and associated binding pocket electrostatic potentials, as depicted in FIG. 1C, is a 4 part process. First, MHC-I structural templates are selected from a database of known MHC-I structures based on their Molprobity score, being a measure of the structure quality. Second, MHC-I variants without existing structures are modeled using the structural template having the best aligning amino acid sequence to that of the MHC-I variant. Third, an ensemble of 40 binding pockets is generated via sidechain rotamer sampling. Fourth, the electrostatic potential of the MHC-I binding pocket is calculated for each ensemble member using APBS software. The ML model is trained on the calculated electrostatic potentials at each position of the binding motif peptide in order to be able to predict an MHC-I binding motif peptide. A schematic of the training of the ML model is depicted in FIG. 1D. As demonstrated by FIG. 1E, the MHC-I binding motif prediction ML model can have many applications such as population analysis, detection of MHC-I binding motif topology, and ligand prediction.

Illustrated in FIGS. 1A and 1B, the MHC-I protein canonically binds peptides of lengths between 8 to 14 amino acids. The second or third and C-terminal residues of the peptide ligand are commonly referred to as ‘anchor’ positions. The identity of the anchor residues remains highly conserved in strong peptide binders. The amino acid distribution at each position of the peptides binding to an MHC-I variant exhibits a consistent amino acid signature, commonly referred to as the peptide binding motif (FIG. 1A). Once presented on the cell surface, the peptide-loaded MHC-I complex interacts with CD8⁺ T cell via the T cell receptor. Stably bound peptide ligands that sufficiently diverge from the naturally presented host peptides, such as those derived from viral or mutated proteins, can trigger an immune reaction.

The cell-specific nature of MHC-I-mediated immune responses is exploited by promising anticancer immunotherapies. However, such therapies rely on the ability to identify peptide targets from an antigen of interest. While recent advances in high throughput tumor immunopeptidome characterization techniques have enabled experimental verification of possible MHC-I targets, the associated costs are still prohibitive for broad clinical application. A major obstacle in predicting the binding of peptides to MHC-I stems from the significant diversity of this protein in the human population. The protein, despite its similar structure, is encoded by one of the most polymorphic genes in the human genome with over 24,000 known sequence variations. The characterization of binding interactions across such diversity proves to be a formidable challenge as even single point mutations in the binding pocket can lead to altered MHC-I peptide binding motifs. In-vitro binding assessments and mass spectrometry-based profiling of cell lines or tumors have provided crucial data for training MHC-I peptide prediction algorithms. Still, binding preferences of the vast majority of MHC-I alleles remain unresolved with the binding data from only 205 variants available in the public databases. Consequently, computational interventions provide a critical solution for identifying potential MHC-I peptides from the antigens of interest.

The diversity of the MHC-I protein was initially tackled through the definition of ‘MHC-I supertypes’ or clusters of MHC-I alleles assumed to produce similar MHC-I binding motifs. Recently, this diversity has been addressed through the development of machine learning algorithms that perform pan-allele sequence predictions. While such sequence-based prediction methods are useful, their binding predictions do not explicitly account for observed physical properties of the MHC-I binding pocket. Therefore, single amino acid changes that may significantly impact the physical environment may be equally weighted with another substitution that preserves the overall state. Attempts to resolve this discrepancy have involved numerically encoding amino acids with molecular properties such as hydrophobicity and the BLOSUM62 substitution matrix scores. Such augmented descriptions of the residues improve the predictive properties of the models; however, they are expected to weakly track with geometric alternations in the binding pocket topology or sidechain orientations arising from MHC-I polymorphism.

Protein-peptide binding is primarily driven by long-range electrostatic interactions. The kinetics of this process is also controlled by the shorter-range interactions, sequence as well as shape complementarity of the binding partners at the interface. Quantitative molecular simulations of such complementarity depend on the accuracy of the molecular model, treatment of protonation equilibria, high-resolution rotamer sampling, geometry optimization, and explicit modeling of the apo and holo states. Despite this knowledge, repeating detailed molecular computations across the allelic diversity of the peptide-MHC-I complexes is extremely expensive, if not prohibitive. The present disclosure simplifies this complex recognition problem into terms of sequence, geometric, and electrostatic variables to elucidate the binding patterns of typical peptide-MHC interfaces from a finite training set of known structures and bonding motifs. Thereafter, the interaction signatures are extrapolated to infer binding across the population of human MHC-I variants.

By analyzing this fundamental immunological process through the lens of molecular electrostatics, this disclosure explores an algorithm for MHC-I binding motif prediction that learns their complementarity relationship with the peptide binders, monitors how MHC-I polymorphisms alter the binding motifs and generalizes computations to a diverse set of alleles. This is accomplished by training an inception-based convolutional neural network (CNN), dubbed “HLA-Inception,” for predicting MHC-I binding motifs based on their correlation with the three-dimensional electrostatic potential distribution of the MHC-I binding pocket. The predicted MHC-I binding motifs are then used to compute peptide binding and stability, study the granularity, and heterogeneity of MHC-I motif networks and analyze phenotypic outcomes to MHC-I associated disease.

### Results

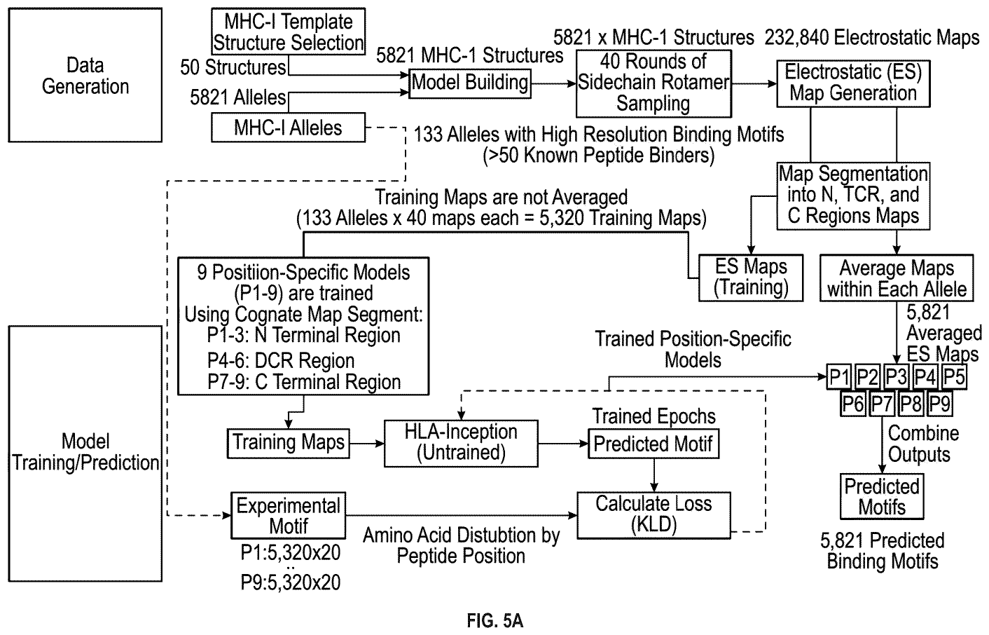

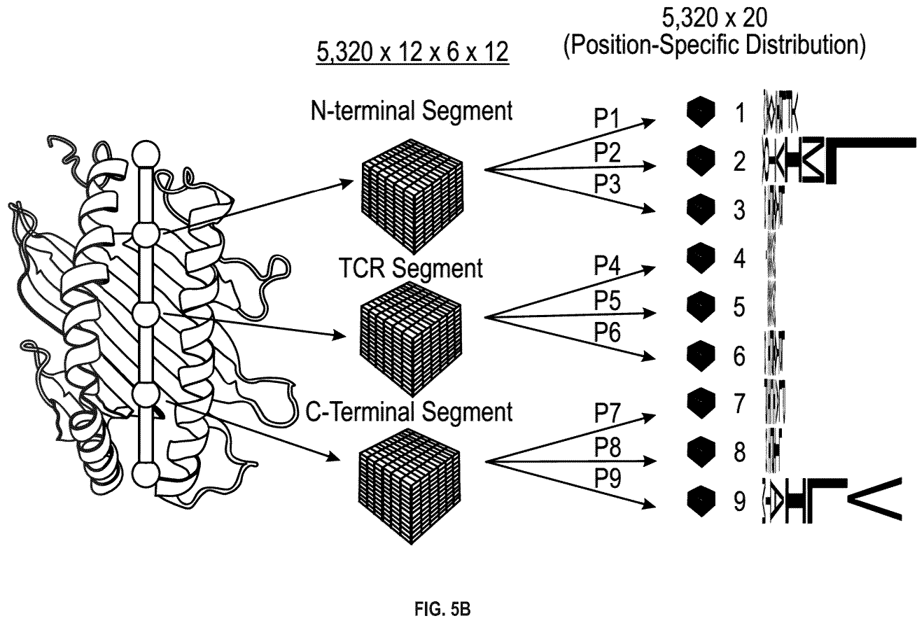

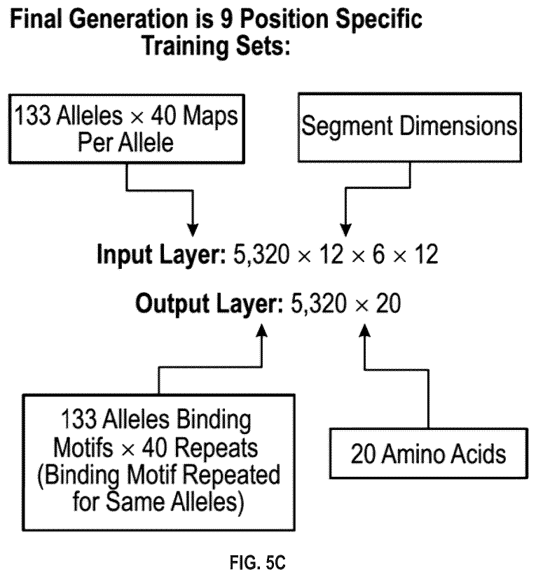

Development of the algorithm for MHC-I binding motif prediction begins with an examination of whether the electrostatic potential of the MHC-I binding pockets bears signatures of their peptide binding motifs or distributions. Using molecular models of 133 unique MHC-I binding pockets, HLA-Inception is then trained to predict the peptide binding motifs of 5,821 MHC variants covering most of the human population based on their electrostatic potentials alone (FIGS. 5A-5C). HLA-Inception is then applied to predicting natural MHC-I ligands and examined disease associations.

### Electrostatic Potential Complements Peptide Binding Motif Variations:

Electrostatic features are central to protein-protein binding. The present disclosure explores these features within the highly polymorphic family of peptide-MHC complexes to seek the collective signatures of peptide binding sequences on the surface of the three-dimensional MHC-I structures. A major roadblock to performing such an analysis of peptide motif diversity is the data available on peptide-MHC interactions, which is sparse relative to the total allelic variance. There are only 133 alleles with binding data known for 50 peptides or greater, and an even more limited number with known structures. So, by generating molecular models of the MHC-I binding pockets, coupled with knowledge on peptide-protein interactions derived form the 133 alleles with known binding motifs, the covariance between pocket features and peptide configuration are tracked. This mapping between binding pockets and peptide configurations sets up an algorithm for grouping the peptide diversity into a small number of functionally relevant binding motifs.

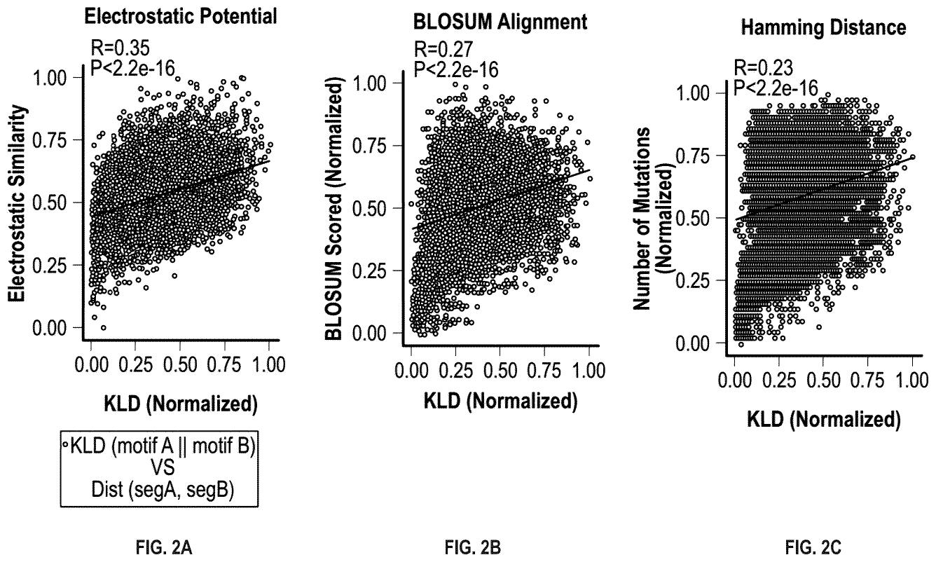

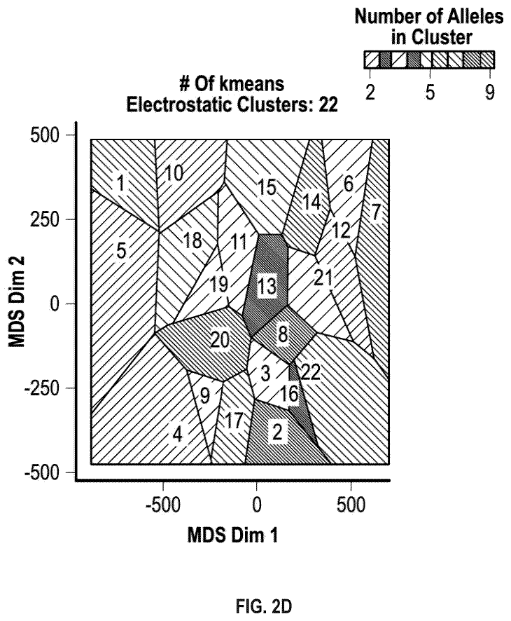

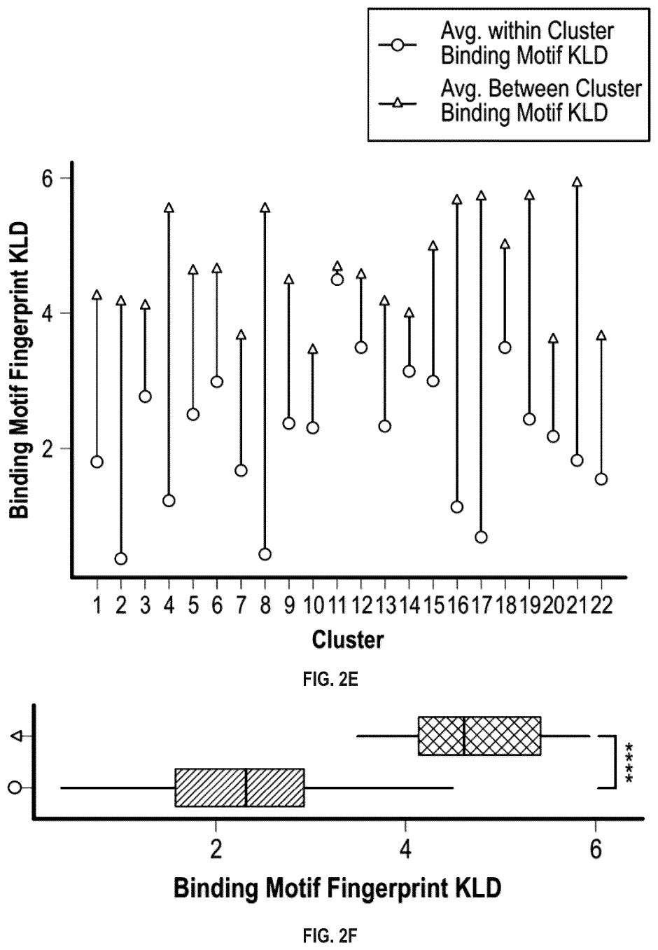

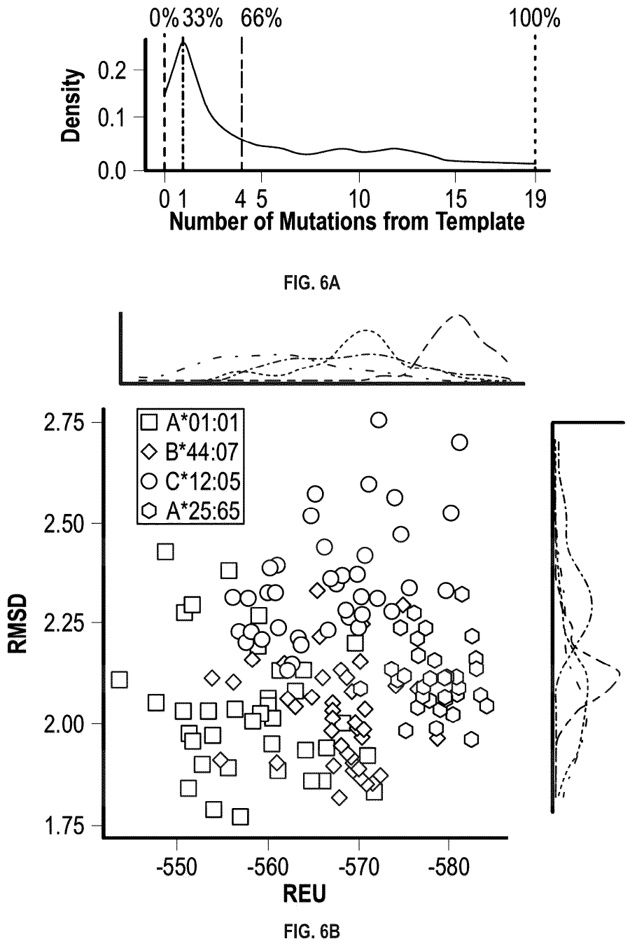

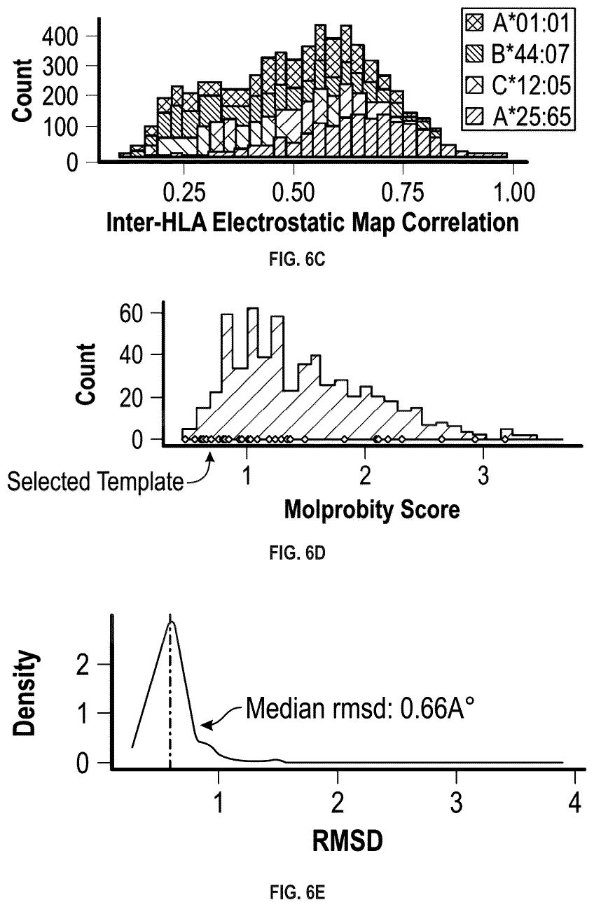

FIG. 1C presents an overview of the multiresolution sequence→molecular ensemble→electrostatics map pipeline, used for generating an exhaustive set of 5,821 MHC-I molecular models. The variance between pocket features and peptide diversity is then tracked using the subset of 133 three-dimensional MHC-I molecular models out of this set (FIG. 6A-E). A metric called the electrostatic potential distance (EPD) is defined for distinguishing between the MHC-I molecular models in the space of electrostatic features as illustrated by Eq. 1, shown below:

$$E P {D}_{xy} = {\sum }_{i = 1}^{n} \sqrt{{( {x}_{i} - {y}_{i} )}^{2}} , \qquad ( 1 )$$

where the i^{th} iso-volumetric voxel occupies the same points in space from two different electrostatic maps corresponding to distinct MHC-I alleles x and y. For the computations in FIGS. 2A-2F, n=2×4/3 π6³≈900 for voxel dimensions of 1 Å³.

MHC peptide binding motif KLD. The similarity of two MHC-I peptide binding motifs, which we call the binding motif KLD, was quantified as the total sum the of observed Kullback-Leibler divergence (KLD) between distributions of amino acids at each position (P1-P9). KLD is a statistical distance measurement that quantifies the divergence of two probability distributions. Kullback-Leibler divergence is calculated using the following equation:

$${D}_{KL} ( P ‖ Q ) = {\sum }_{i = 1}^{k} {P}_{i} log \frac{{P}_{i}}{{Q}_{i}} . \qquad ( 2 )$$

In the above equation, P and Q are discrete probability distributions with k number of bins. Due to the non-symmetry of KLD calculation, i.e., DKL (P∥Q)=/DKL (Q∥P), the KLDs reported in this paper represent a symmetrized KLD computed by finding the average of the KLD values calculated in both directions. The equation is as follows:

$$K L D = \frac{{D}_{KL} ( P ‖ Q ) + {D}_{KL} ( Q ‖ P )}{2} . \qquad ( 3 )$$

An EPD between two MHC-I variants represents the Euclidean distance between two sets of binding pocket potential volumes. The electrostatic potential volumes were extracted near the N- and C-terminal binding pockets and the pairwise EPDs were computed between all the 133 alleles. Similarly, to describe the diversity between peptide motifs (with 50 or more known binders) that are already known to bind these MHC-I pockets, the inter-motif Kullback-Leibler divergence or binding motif KLD was computed by Eq. 4, shown below:

$$Binding Motif K L D = {\sum }_{i = 1}^{g} K L {D}_{i} . \qquad ( 4 )$$

As depicted by the plots in FIGS. 6A-6E, the utilized protein modeling method is robust to multiple point mutations. The overall distribution of the number of mutations to select template structures required to generate protein models of all 5,821 MHC-I alleles is depicted in FIG. 6A. The highlighted alleles (A*01:01, B*44:07, C*12:05,A*25:65), correspond to the 0^{th}, 33^{rd}, 66^{th}, and 100^{th} percentile in the number of required mutations, respectively. A scatter plot of the sidechain RMSDs and overall energy of the structure in Rosetta energy units of each highlighted allele following the sidechain rotamer sampling protocol is depicted in FIG. 6B, which also demonstrates the marginal distributions for each dimension. A total of 606 MHC-I structures were considered, the overall distribution of their Molprobity scores are plotted in FIG. 6D.

Correspondence between the MHC-I EPD and binding motif KLD across the reference set of models, illustrated in FIGS. 2A-2C, reveal a linear correlation between MHC-I and binding peptide motif diversity (R=0.35). Each dot depicted indicates a pairwise comparison between two alleles with the binding pocket distance metric defined by the electrostatic potential, Hamming distance, or BLOSUM alignment. As controls, two sequence-dependent distance metrics, namely the Hamming distance and BLOSUM62 alignment, were also employed to perform inter-allele comparisons. While the Hamming distances track weakly with the binding motif KLD, the more biochemically aware BLOSUM62 alignment improved the correlation coefficient from 0.23 to 0.27. Taken together, the incorporation of physical volumetric data beyond sequence information better tracks MHC-I protein changes with substantive alterations to the peptide binding motif.

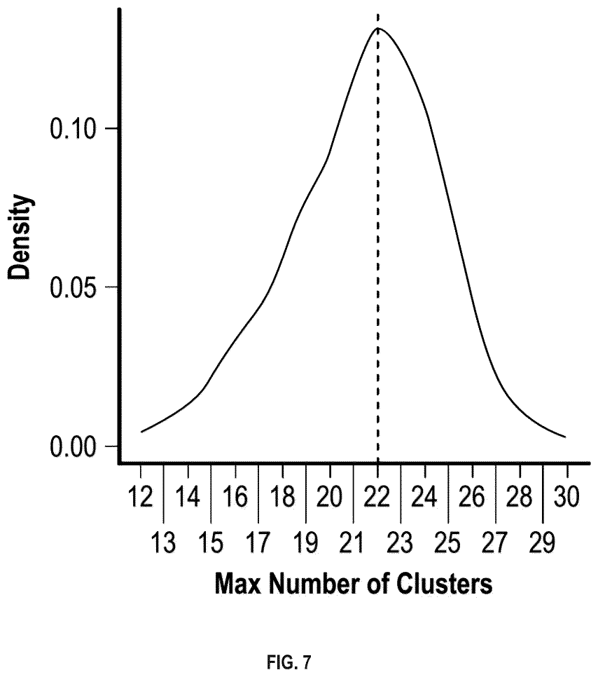

Building on the covariance between MHC-I EPD and binding motif KLD, whether the electrostatic description of MHC-I protein can be employed to classify their peptide binding motifs was explored. Spherical regions of electrostatic potential corresponding to the N- and C-terminal anchor binding pockets were extracted, after which the K-means clustering method was applied to find the optimal number of clusters where each cluster had at least 2 assigned alleles. Multidimensional scaling was performed on the concatenated electrostatic data and the position of each cluster was defined as the arithmetic average of cluster members. The reduced space was then visualized with a Voronoi diagram. Each Voronoi cell is labeled with the respective cluster number and the fill color indicates the number of alleles assigned to each cluster. A K-means clustering performed on the electrostatic potential grids extracted for EPD calculation revealed 22 unique clusters (FIGS. 2D, 7). The average binding motif KLD between alleles within the same cluster identified in FIG. 2D was compared to the pairwise binding motif KLDs of every allele outside that cluster. Notably, the MHC-I variants with comparable peptide binding motifs are grouped together, with the average separation within their clusters being less than those between the clusters (FIGS. 2E and 2F). Therefore, the electrostatic potential features analyzed across a set of MHC-I proteins can be indicative of the signature distributions of amino acids that compose a peptide binding motif.

### Inception Model Trained on Electrostatic Features Detects the Heterogeneity of Binding Motif

While the EPD values successfully tracked with variations in MHC-I binding motifs, such analysis is inherently limited by the paucity of experimental data. To address this limitation, a deep learning model, named HLA-Inception, was developed. This CNN model is trained on three-dimensional representation of the electrostatic potential within the MHC-I binding pocket (FIG. 1D) to reproduce known peptide binding motifs. Following training, the neural network was applied to the generated structures to estimate binding motifs for MHC-I structures with unknown peptide interaction signatures.

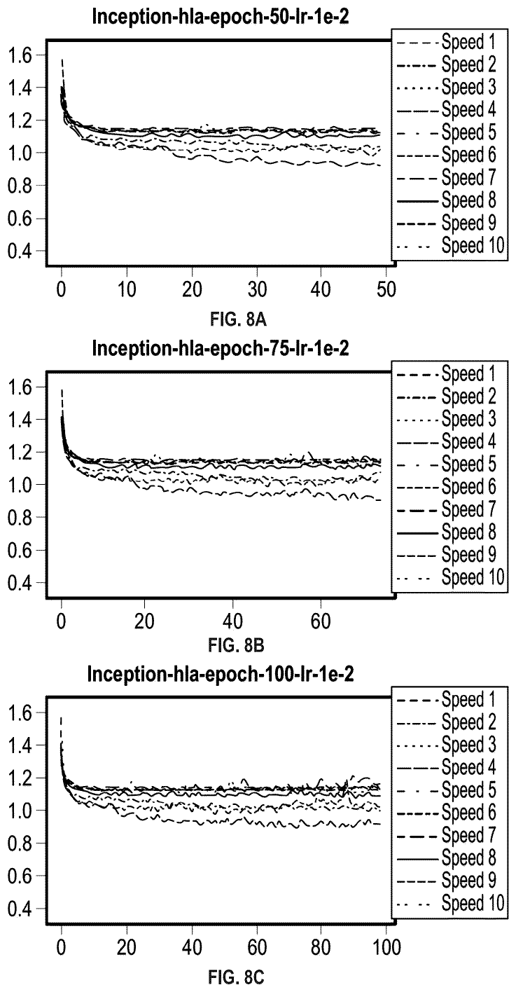

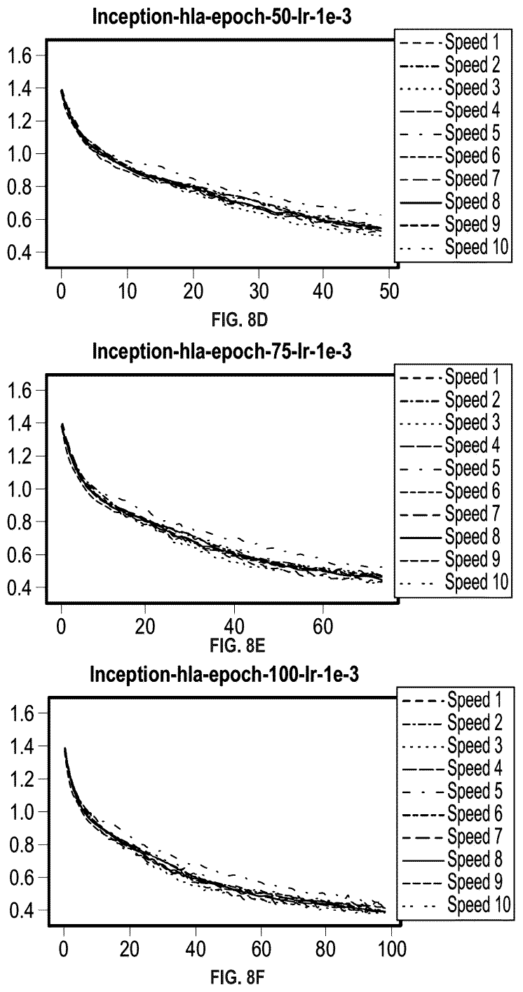

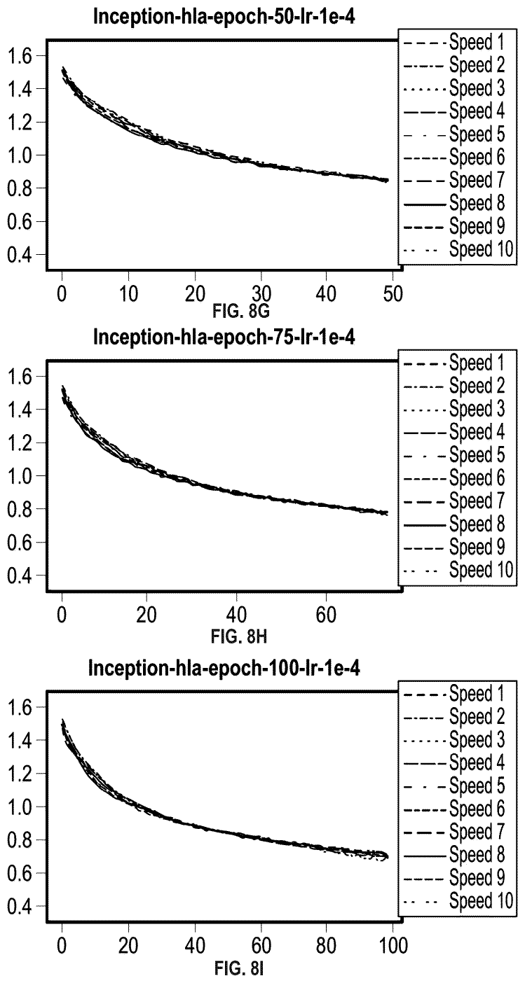

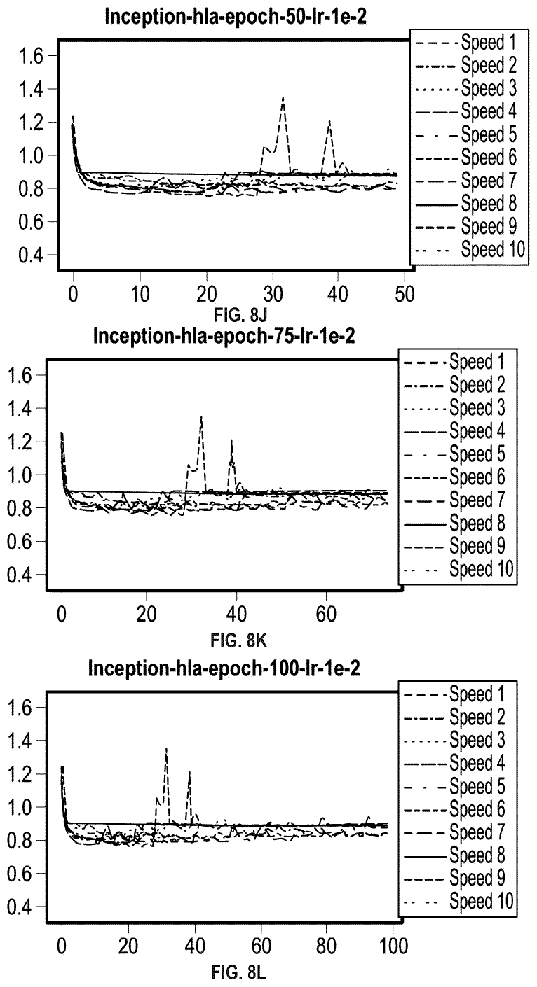

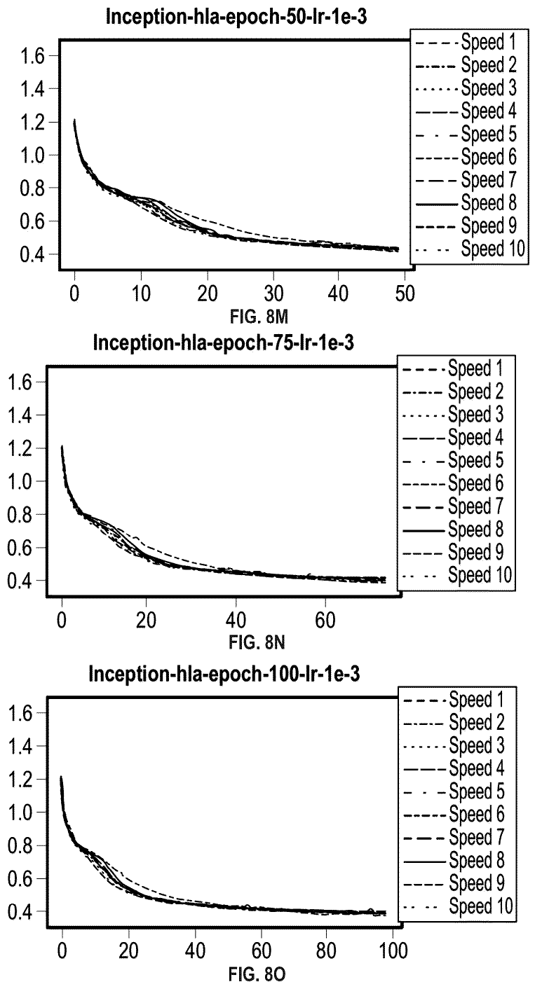

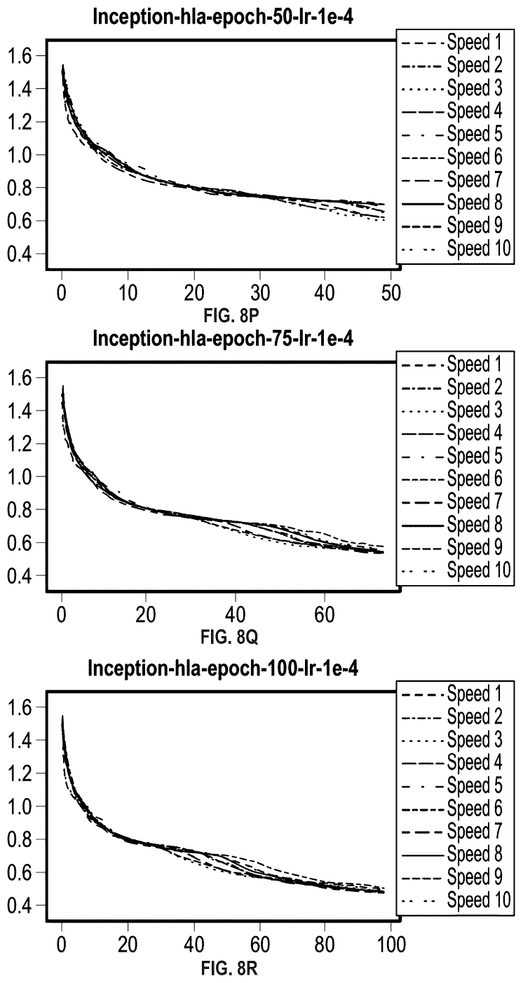

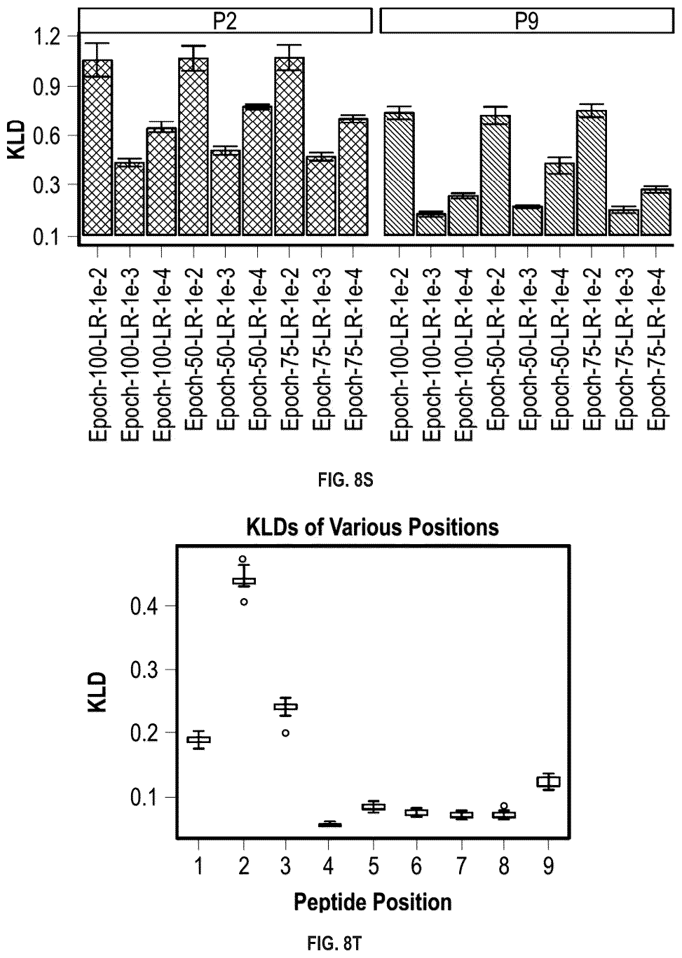

The approach disclosed herein works by segmenting an MHC-I binding pocket electrostatic potential grid into a number of volumes. Here, three equal-sized volume segments of the peptide binding pocket were chosen—the region corresponding to N-terminal binding pocket, the TCR contact region, and the C-terminal binding pocket. The N-terminal segment was used to predict amino acid distribution for peptide positions 1-3; the TCR contact segment monitors positions 4-6; and the C-terminal segment probes positions 7-9. The segmented electrostatic volumes of the MHC-I pocket at each of these peptide positions are passed through the CNN, using an output layer of amino acid distributions at the respective peptide positions (P1-P9). Using the training set of 133 MHC-I binding pocket models with experimentally verified peptide binding motifs, each CNN was trained to replicate the known amino acid distribution at a specific peptide position. Due to the overall importance of positions 2 and 9 to peptide binding, hyperparameter tuning was focused on these positions (FIGS. 8A-8T). After several epochs of minimizing the loss between empirical and predicted position-specific distributions, the peptide binding motif is determined when all nine CNNs converge.

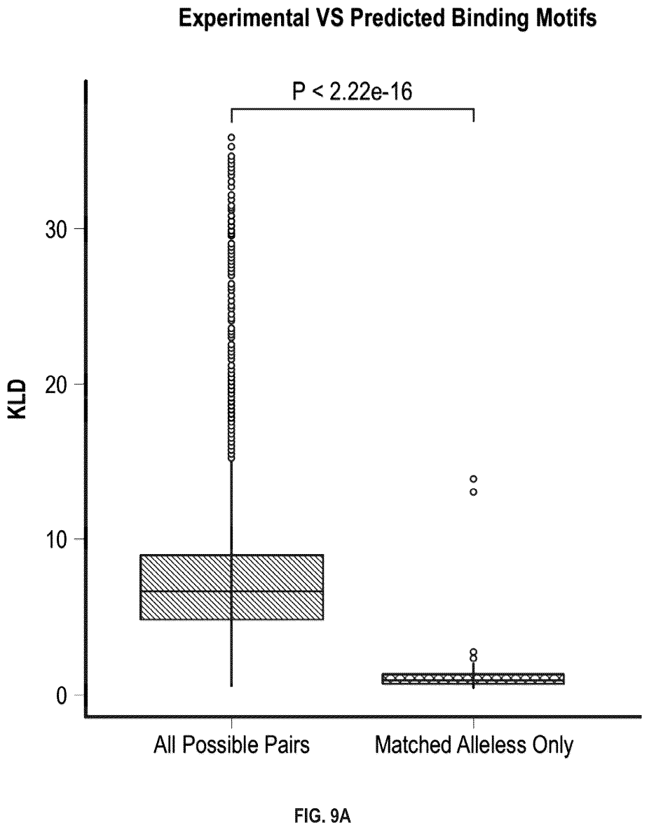

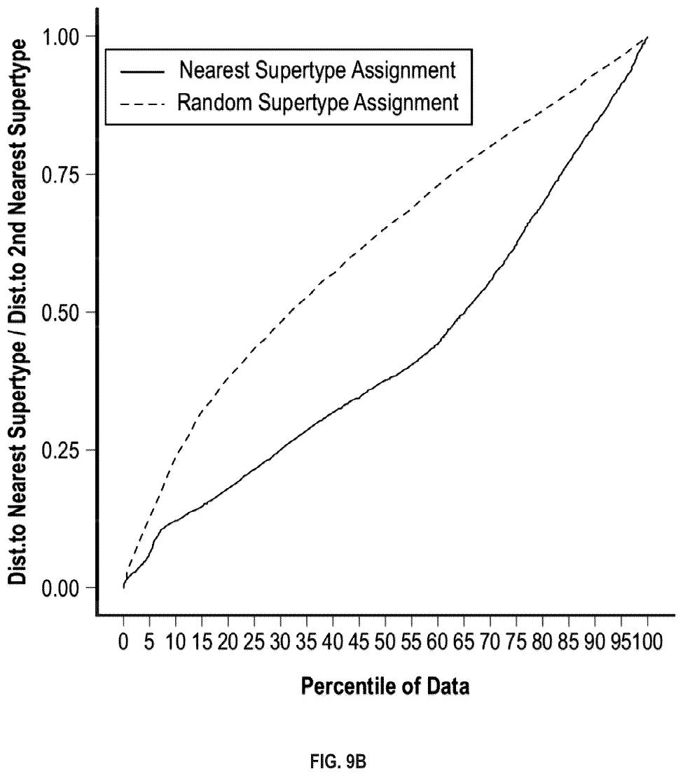

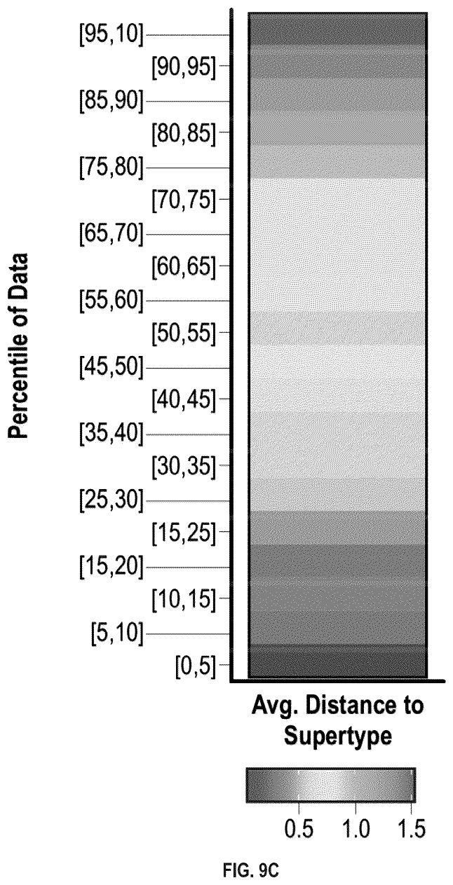

Following model training with 5,320 unique maps (ensemble of 40 conformations/allele X 133 alleles with known binders), the HLA-Inception model was applied to the average electrostatic potential maps from all generated MHC-I structures (FIGS. 1C-1E, 5A-B). As an initial check of quality, it was found that the binding motifs predicted from the average electrostatic maps of the 133 training were in close agreement with the experimentally determined peptide binding motifs (FIG. 9A). Thereafter, using the binding motifs data from only these 133 variants, the disclosed model assigned all the 5,821 MHC-I alleles.

Referring to FIGS. 9A-9C, the KLD of each predicted binding motif was measured and compared to its corresponding experimentally resolved binding motif. To give this KLD value context, the total motif KLD was calculated from random pairs of experimental and predicted binding motifs. The confidence of the supertype assignment of each predicted allele was also measured by calculating the ratio between the assigned supertype with lower values indicating more confident supertype assignments. The alleles were then ordered by the ratio of these distances and converted to percentiles of all alleles. For purposes of comparison the distance ratio was recalculated using a random supertype assignment. The alleles that were grouped using the nearest supertype strategy were binned by percentile and the average distance was calculated. This revealed that on average alleles with low confidence of supertype assignment (high distance ratio) tended to be further from their assigned supertype, indicating that these alleles might be better assigned using additional novel supertypes.

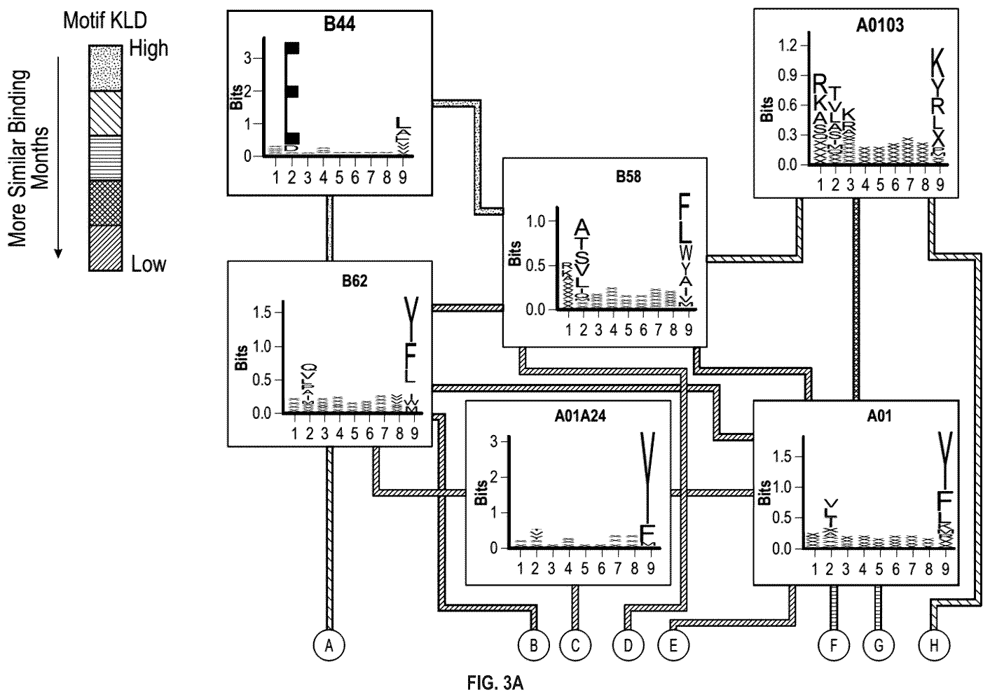

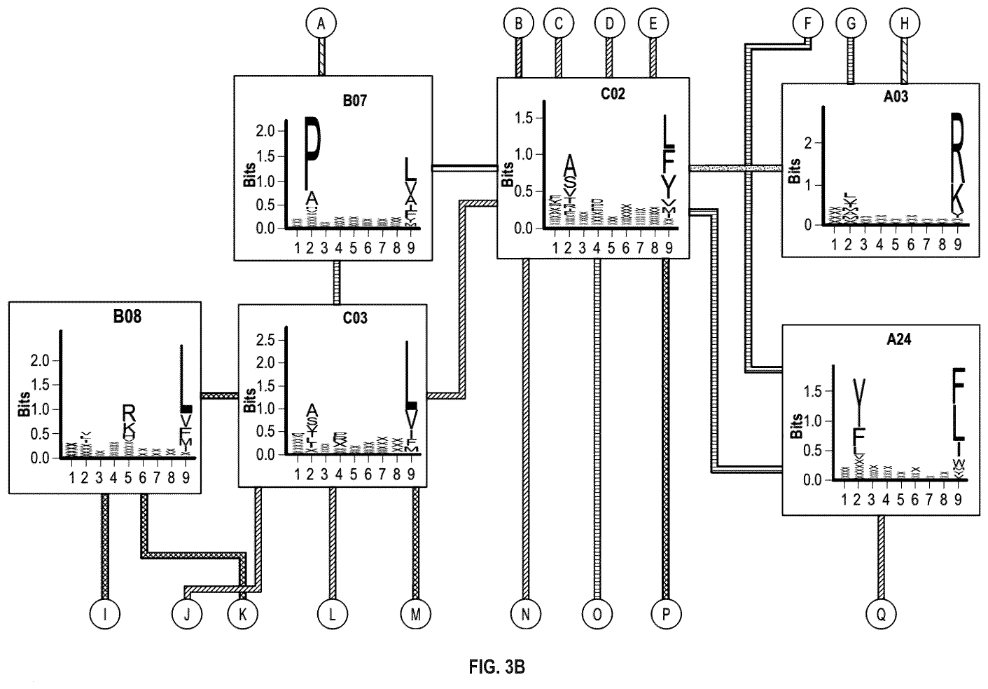

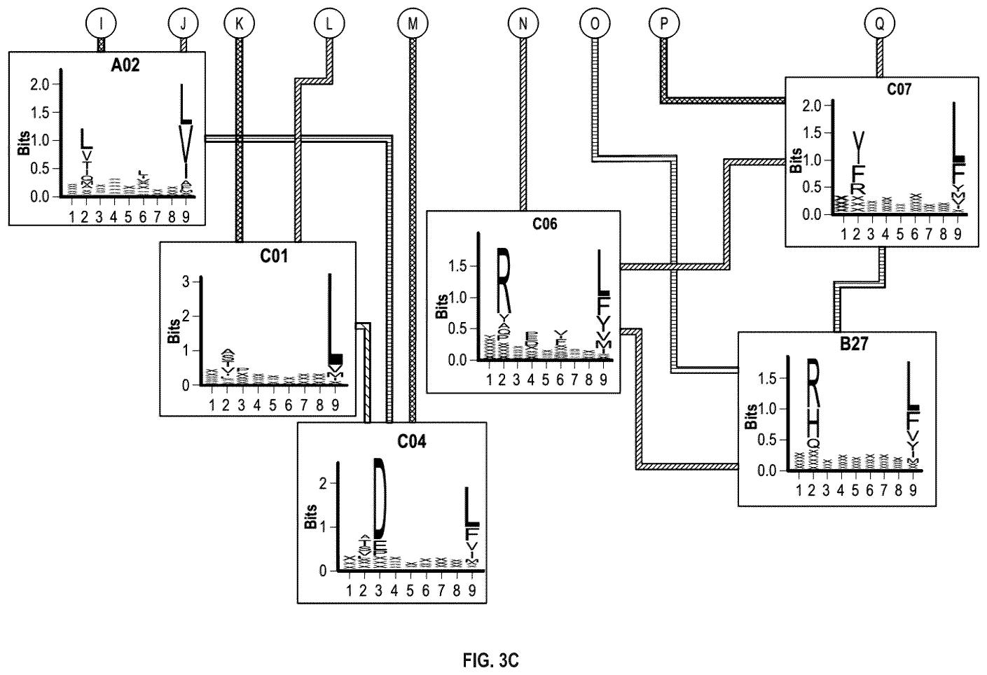

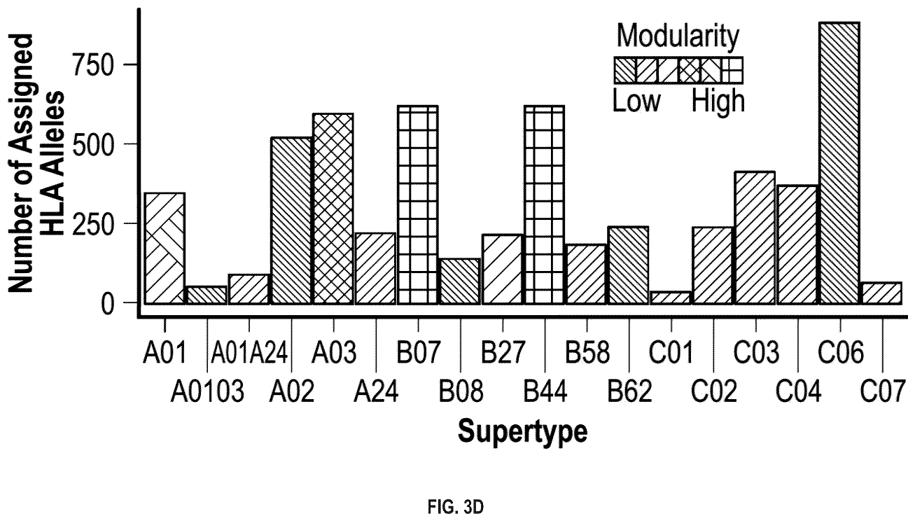

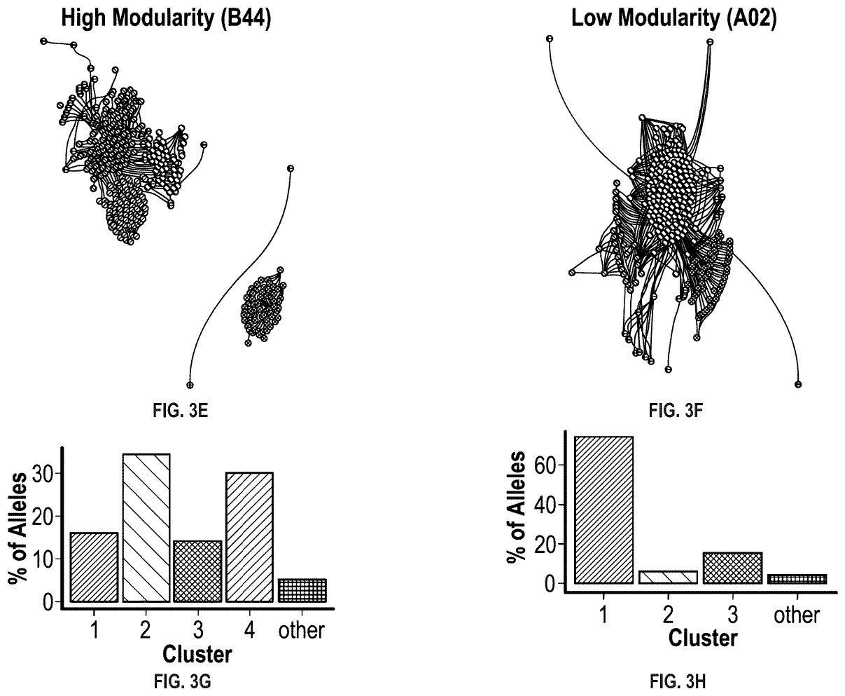

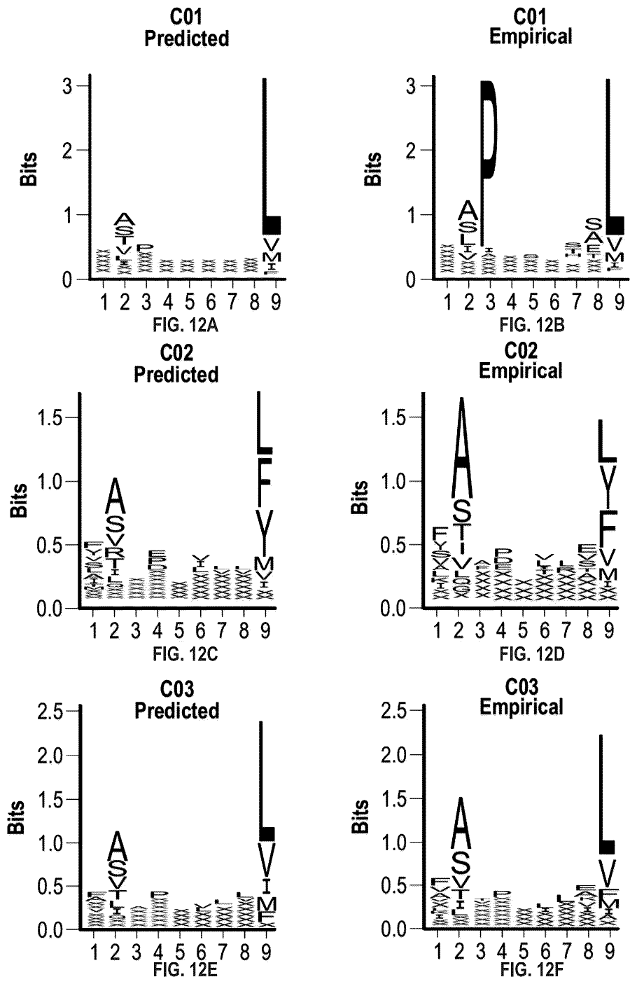

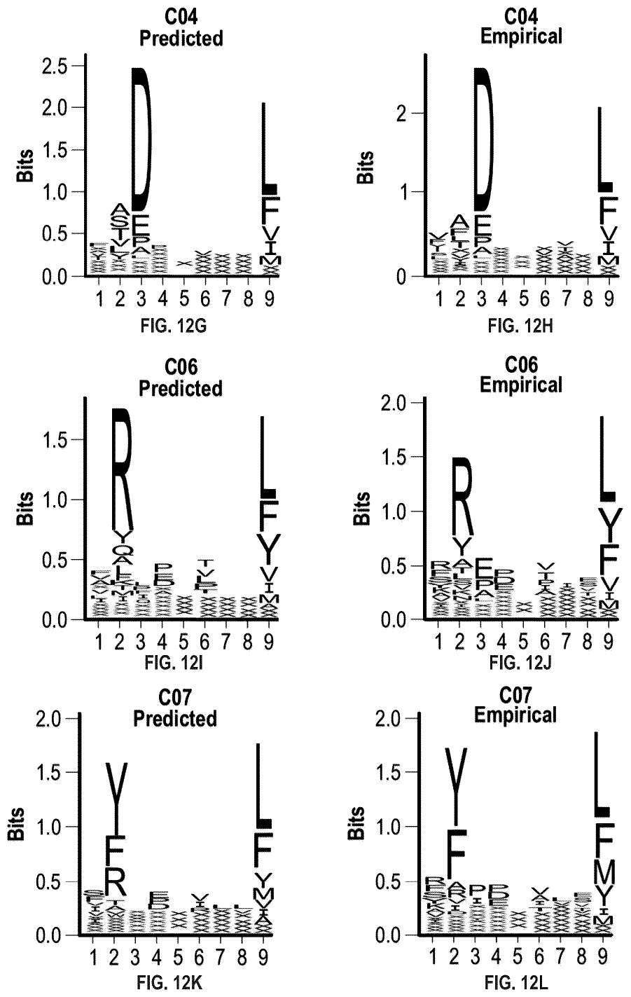

The macroscopic arrangement and structure of the MHC-I binding motif space was investigated using a force-directed graph, where each node represents a different MHC-I supertype19 and an edge represents the minimum KLD between the binding motifs of the corresponding supertypes (FIGS. 3A, 10A-12L). Each node was then connected to the 3 most similar MHC-I supertye binding motifs as determined by binding motif KLD with any edge defining a binding motif KLD of ≥4 also being removed. Warm colors on the network depicted in FIGS. 3A-3C indicate dissimilar binding motifs, while cooler colors indicate more similar motifs. Supplementing the traditional supertype classifications (945 alleles), we assigned each of the 5,821 MHC-I alleles to an MHC-I supertype based on the minimum KLD between the HLA-Inception predicted binding motif and that of a supertype node in FIG. 3A. Following graph generation, the heterogeneity of submotifs within each supertype was explored through community structure analysis. MHC-I supertypes marked by high submotif heterogeneity indicate a larger number of distinct binding submotifs while low heterogeneity supertypes suggest fewer and more homogeneous submotifs. The number of the 5,821 MHC-I alleles that were assigned to each MHC-I supertype is depicted in a bar plot shown in FIG. 3D. All alleles assigned to a given supertype were determined using the infomap algorithm discussed in the Methods section below. The modularity of each supertype graph, a measure of connection density within each community, is indicated by the color of the bar. Remarkably, the population or size of an MHC-I supertype does not always imply its heterogeneity (FIG. 3D, 13A). While populated supertypes like B07 and B44 represent heterogeneous communities of motifs, a similarly-sized A02 node offers a much more homogeneous distribution (FIGS. 3E-3H). Similarly, the more populous C06 augmented supertype is in fact less heterogeneous than B07 and B44, and a smaller sized A01 supertype is more heterogeneous than the larger A02.

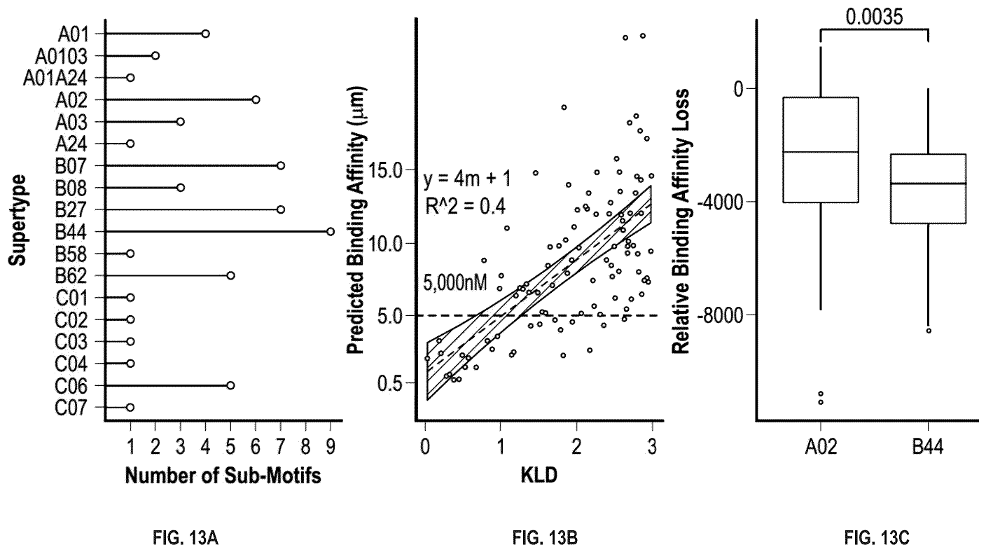

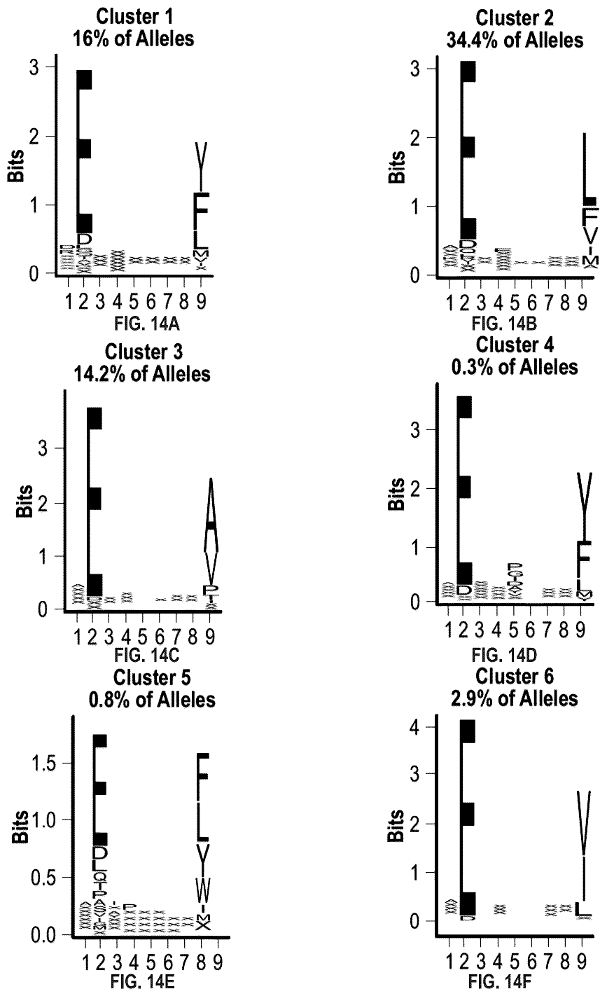

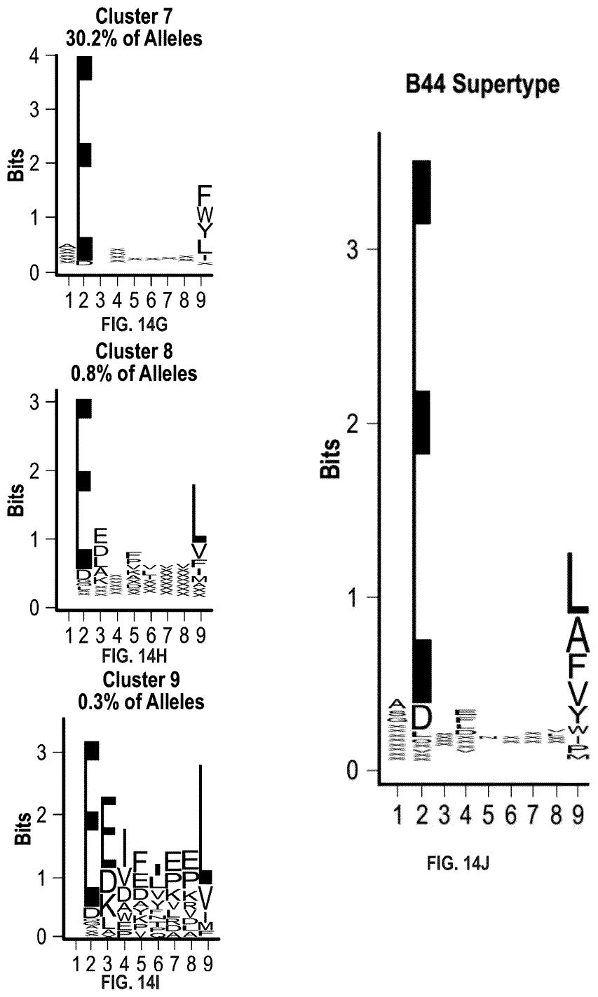

Referring to FIGS. 13A-14J, lollipop plots for the A02 and B44 submotifs are shown, demonstrating the number of sub-motifs identified within each supertype. Peptides with known binding affinity to an allele assigned to the A02 or B44 supertype were randomly sampled and the binding affinity of each peptide was tested with respect to other alleles within that supertype. Using the infomap community detection algorithm, the sub-motifs with alleles assigned to the A02 supertype were identified and the percentage of alleles that were within the given sub-motif are depicted in the percentages below each sub-motif cluster. The overall binding motif of that cluster was determined by the average binding motifs of the clustered alleles. Similar analysis for the B44 analysis can be seen in FIGS. 14A-14J.

MHC-I supertypes are important for generalized vaccine development. Based on the classification utilized herein, the supertypes marked by high heterogeneity (e.g., B44) exhibit a broad and more distinct range of peptide binding sub-motifs, while the homogeneous supertypes (e.g., A02) have a sharper distribution across similar numbers of member MHC-I alleles (FIGS. 13B and 13C). Importantly, within supertypes of high heterogeneity, there is a significantly larger loss of average predicted binding affinity when performing intra-supertype cross-allele binding predictions that are not observed in more homogeneous supertypes (FIG. 13C). This observation of supertype heterogeneity will have important implications for supertype-level peptide vaccine design as therapeutic peptides targeted to bind to supertypes with high heterogeneity might not equally cover all member alleles. Taken together, the electrostatic augmentations to classical supertype via HLA-Inception bring to light unforeseen topological details of peptide-MHC complexes.

Integration of Electrostatics with Sequence Information Offers Accurate Pan Allele MHC-I Peptide Ligand Prediction

The extreme polymorphism of the MHC-I protein typically results in the need to identify peptide targets for MHC-I alleles without experimentally resolved peptides. This need has brought forth pan-allele prediction algorithms, which leverage information from alleles with known binders to extrapolate to the unknown ones. Here, pan-allele peptide prediction is performed by employing the peptide binding motifs derived from HLA-Inception to define a position-weighted matrix (PWM) scoring system.

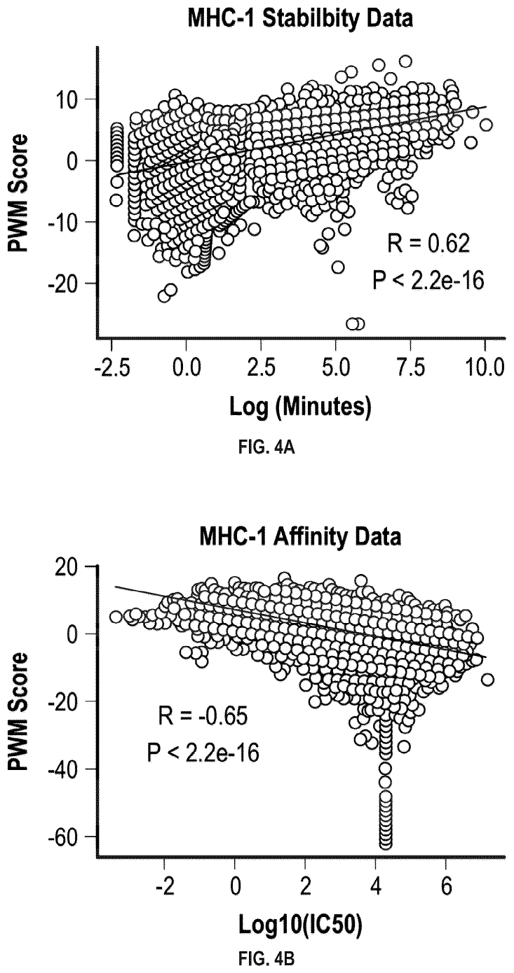

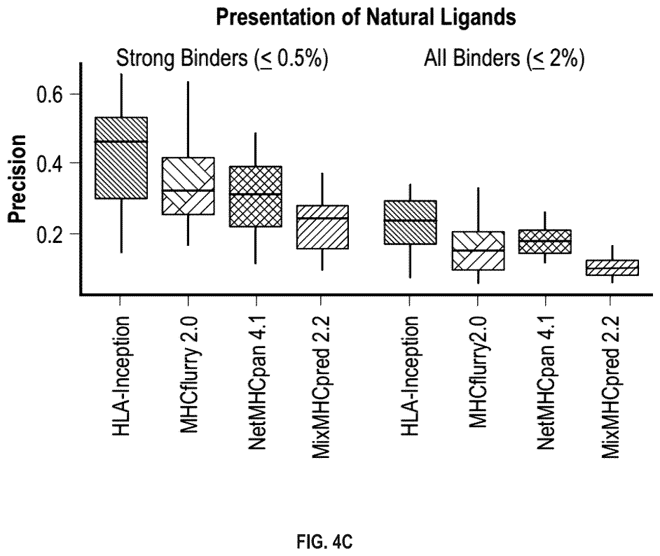

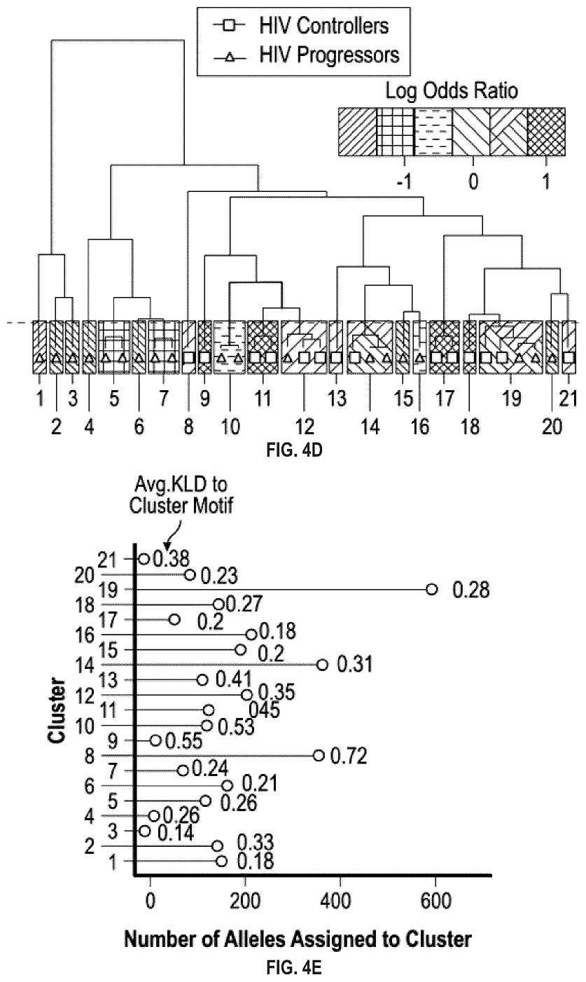

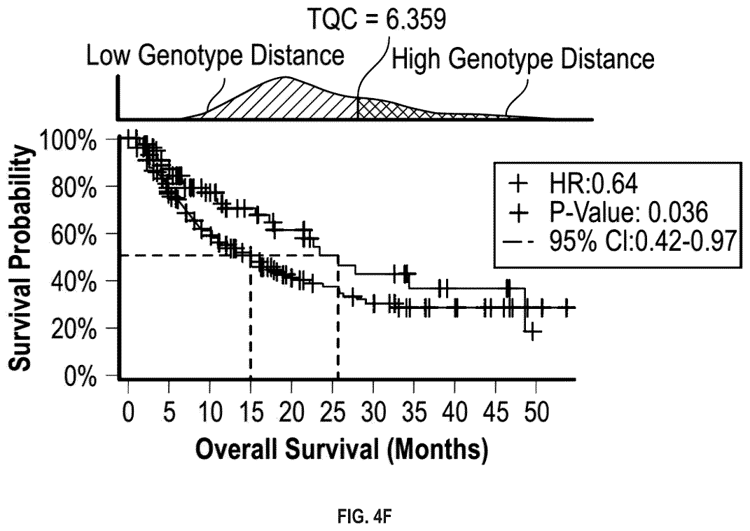

PWM score utilizes log-odd ratios of observing an amino acid at a particular position to determine how well a peptide fits a binding motif for a given allele. Therefore, peptides characterized with a high PWM score are implied to have a high probability of stable binding. To assess PWM as peptide binding metric, peptides with quantitative binding estimates, namely peptide binding affinity (IC50) and MHC-I stability (minutes), were ranked based on the PWM score. We found that PWM scores were strongly associated with binding estimates, this correlation can be seen in FIGS. 4A and 4B with each point representing a different peptide that was tested. PWM scores had an absolute correlation coefficient of 0.62 with MHC-I stability data, and a 0.65 correlation with MHC-I affinity. This result is particularly notable as it suggests that the most probable binders determined from the disclosed algorithm are also found to be strong binders, even though no quantitative protein-peptide interaction data was used to train the model. The 60-65% correlation indicates that the inception network has learned to capture the strengths of their molecular interaction with the MHC-I binding pocket, leveraging only information about the MHC-I binding pocket environment and overall binding motif of the peptides.

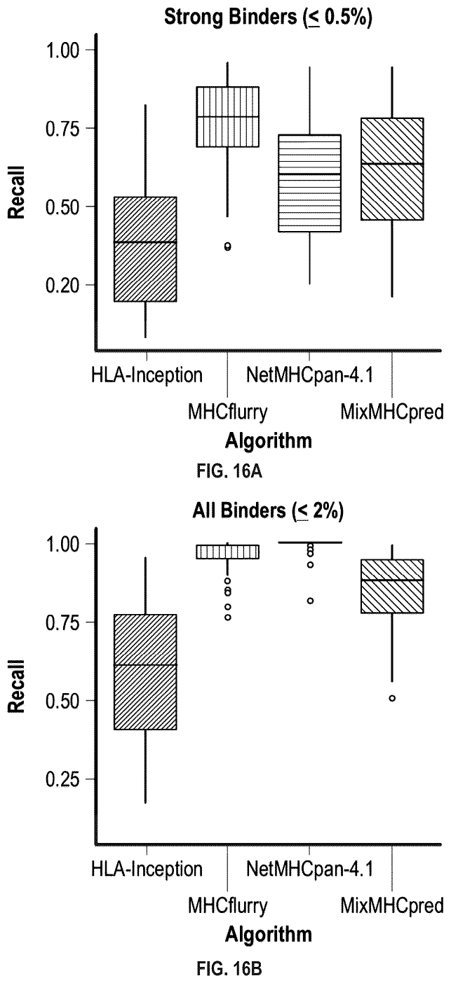

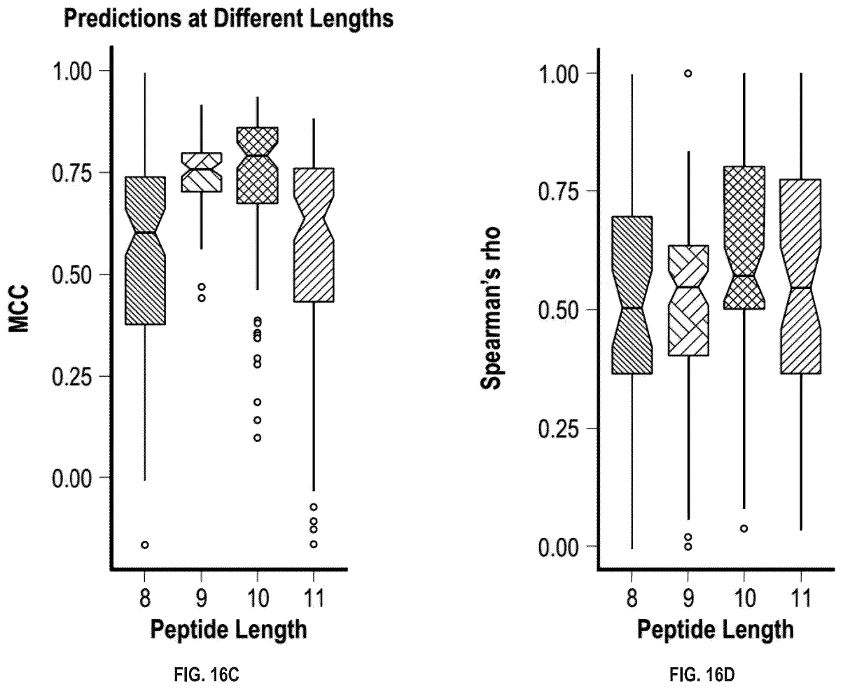

The precise identification of peptides that are likely to be naturally presented on the cell surface has significant clinical value for the development of T cell-based immunotherapies. However, this task necessitates the consideration of many potential peptides where the number of presented peptides are severely outnumbered by the number of possible peptides, often leading to high false positive rates. Therefore, an examination of the precision of HLA-Inception on the recovery of naturally presented peptide ligands was performed. The target peptides used for this analysis were extracted from a data set of MHC-I peptides determined by mass spectrometry to be naturally presented and were combined with an excess of decoy peptides that were not detected by mass spectrometry, resulting in a final target-decoy ratio of 1:100. The performance of HLA-Inception-based predictions was compared to several of the current state-of-the-art for MHC-I peptide prediction algorithms: MHCflurry2.0, NetMHCpan-4.1 and MixMHCpred2.2. Peptide selection was based on two commonly used score thresholds, 0.5% and 2%, where peptides with this score or lower can be considered to bind stronger than 99.5% and 98% of all possible peptides respectively. Accordingly, precision was measured relative to the 0.5% and 2% binding percentile cut off thresholds which selected for strongly binding and all binding peptide respectively. It was found that HLA-Inception performance achieved a median precision of 0.46 and 0.234 for the 0.5% and 2% threshold, respectively (FIG. 4C). While the other algorithms achieved better recall (FIGS. 16A and 16B), they had significantly lower median precision values (1.42-2.34 fold precision increase). This result is significant, as the other algorithms were explicitly trained on portions of the data within this testing set, whereas HLA-Inception predictions only used an overall peptide-binding motif. Furthermore, the relatively simple mathematical operations underlying HLA-Inception-based peptide prediction enable proteome-scale binding prediction in a matter of seconds (Table 1).

TABLE 1
Prediction timing. HLA-Inception was used to identify binding
peptides from the complete human and mouse reference
proteomes as indicated in the first column. All
predictions were done with respect to nonameric HLA-A02:01
peptides that were predicted to binding in the top 0.5% percentile.

| | | Total 9mer | Binding | Predicted | Prediction |
|---|---|---|---|---|---|
| Proteome | Proteins | Peptides | threshold (%) | Binders | time (s) |
| Human | 81,837 | 29,049,213 | 99.5 | 148,892 | 5.73 |
| | | | 98 | 590,201 | 9 31 |
| Mouse | 55,286 | 22,844,078 | 99.5 | 121,228 | 4.64 |
| | | | 98 | 481,594 | 7.59 |
| Virus | 3,383 | 1,385,612 | 99.5 | 7,678 | 1.93 |
| | | | 98 | 30,714 | 2 13 |

Referring to FIGS. 14D-14F, predicted binding motifs for MHC-I alleles associated with HIV control or progression were hierarchically clustered. The clusters were then determed by cutting th tree at a binding motif KLD of 1. The colored bars under the tree in FIGS. 4D and 4E indicate each cluster with color indicating the average log odds ratio of being an HIV controller based on alleles assigned to that cluster. The lollipop plot depicted indicates the number of alleles assigned to each cluster based on binding motif KLD distance with the color indicating the average log odds ratio. The genotype distances between the alleles associated with HIV control or progression and the predicted binding motif were calculated for patients receiving immune checkpoint inhibitors. Patients exhibiting genotype distances within the top quartile of all patients were designated as high genotype distance individuals while the remaining patients were assigned to the low genotype distance group. The Kaplan-Meier plots depicted in FIG. 4F were constructed for both groups.

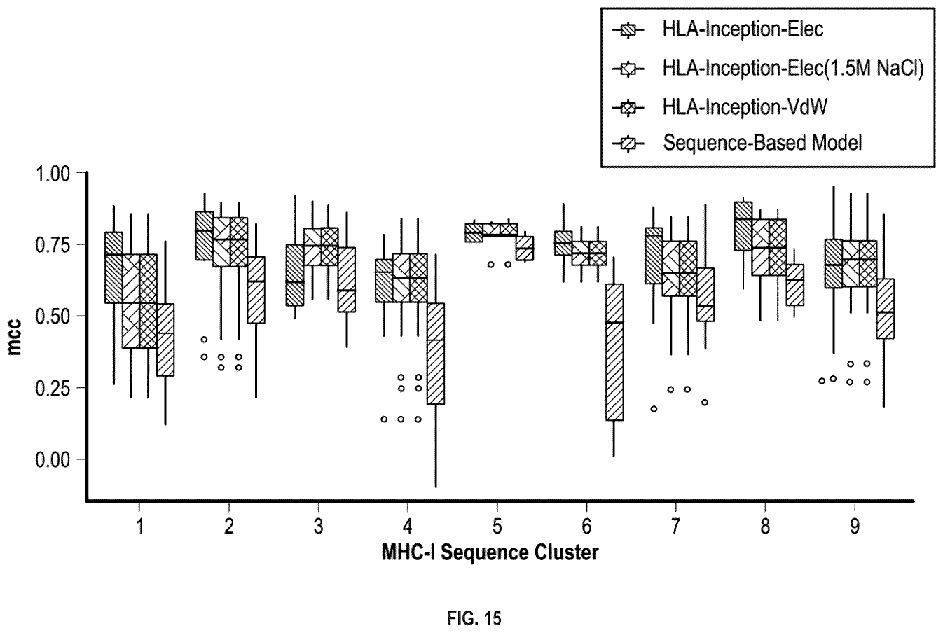

Ultimately, the true value of any pan-allele prediction lies in its ability to be extrapolated to unseen MHC-I alleles, a setup in machine learning referred to as “zero-shot prediction”, which can be quantified using ‘leave-one-out’ cross-validation analysis. In this analysis, data corresponding to a target allele is removed from the training set, and then the remaining data is used to train the algorithm. The accuracy of the algorithm is determined by testing the withheld data. However, such validation approaches fail to account for the existence of highly homologous MHC-I alleles still contained within the training set. The inclusion of homologous alleles has the potential to artificially boost algorithm performance. A more rigorous test of pan-allele predictive properties can be performed by ensuring that highly homologous alleles are removed prior to training and these allele groups are collectively tested to assess the generality of the algorithm. To that end, the binding pocket sequences for the 133-allele set were clustered using BLOSUM62 alignments, where each allele was assigned to a cluster of alleles with similar amino acid sequences. The alleles that were assigned to each sequence cluster as determined by k-menas clustering of BLOSUM62 encoded sequences are listed in Table 2. In order to appropriately benchmark the performance of HLA-Inception peptide predictions with a sequence-based approach, a neural network trained on these BLOSUM62 encoded peptides and key binding pocket residues was built. Using the MHC-I binding pocket sequence clusters, leave-one-cluster-out analysis was performed using both neural networks (FIG. 15). It was discovered that electrostatics-based binding classification predictions produced a median Matthews correlation coefficient (MCC) of 0.72 (IQR: 0.59-0.79), while the sequence-based model produced a median MCC of 0.52 (IQR: 0.38-0.68), suggesting a 38% improvement over a sequence-based prediction method. The MCC was calculated with respect to peptide prediction using an HLA-Inception model trained on electrostatics, an HLA-Inception model trained using electrostatics maps with a high salt concentration, an HLA-Inception model trained on van der Waals maps, and a sequence-based model. Each of these calculations can be found plotted and compared in FIG. 15. This improvement indicates that the incorporation of electrostatics improves universal peptide prediction. To verify that the network has indeed leamed the electrostatic signals for predicting the complementary peptide motifs, all the 5,320 maps were recomputed at a higher salt condition, wherein the map features are washed out (FIG. 15). Indeed, for the majority of the peptide motifs the MCC decreased, but still out-performing sequence-only predictions.

TABLE 2
Alleles tested in each cluster

| | |
|---|---|
| Cluster 1 | A_01:01 |
| | A_03:01 |
| | A_11:01 |
| | A_11:02 |
| | A_29:02 |
| | A_30.01 |
| | A_30:02 |
| | A_31:01 |
| | A_32:01 |
| | A_32:07 |
| | A_32:15 |
| | A_33:01 |
| | A_33:03 |
| | A_34:02 |
| | A_36:01 |
| | A_74:01 |
| | A_80:01 |
| Cluster 2 | B_27:01 |
| | B_27:02 |
| | B_27:03 |
| | B_27:04 |
| | B_27:04 |
| | B_27:06 |
| | B_27:07 |
| | B_27:08 |
| | B_27:09 |
| | B_27:20 |
| | B_40:01 |
| | B_40:02 |
| | B_40:06 |
| | B_40:13 |
| | B_48:01 |
| Cluster 3 | B_07:02 |
| | B_07:04 |
| | B_08:01 |
| | B_14:02 |
| | B_18:03 |
| | B_39.01 |
| | B_39:24 |
| | B_42:01 |
| | B_54:01 |
| | B_55:01 |
| | B_55:02 |
| | B_56:01 |
| Cluster 4 | A_02:01 |
| | A_02:02 |
| | A_02:03 |
| | A_02:04 |
| | A_02:05 |
| | A_02:06 |
| | A_02:07 |
| | A_02:11 |
| | A_02:12 |
| | A_02:16 |
| | A_02:17 |
| | A_02:19 |
| | A_23:01 |
| | A_24:02 |
| | A_24:03 |
| | A_24:07 |
| | A_24:13 |
| | A_69:01 |
| Cluster 5 | C_01:02 |
| | C_06:02 |
| | C_12:03 |
| | C_14:02 |
| | C_14:03 |
| | B_16:01 |
| Cluster 6 | B_46:01 |
| | C_02:02 |
| | C_03:02 |
| | C_03:03 |
| | C_03:04 |
| | C_04:01 |
| | C_04:03 |
| | C_05:01 |
| | C_07:01 |
| | C_07:02 |
| | C_07:04 |
| | C_07:06 |
| | C_08:01 |
| | C_08:02 |
| | C_12:02 |
| | C_15:02 |
| | C_15:05 |
| Cluster 7 | A_25:01 |
| | A_26:01 |
| | A_26:02 |
| | A_26:04 |
| | A_26:08 |
| | A_34:01 |
| | A_66:01 |
| | A_68:01 |
| | A_68:02 |
| | A_68:23 |
| Cluster 8 | B_37:01 |
| | B_41:01 |
| | B_41:03 |
| | B_41:04 |
| | B_44:02 |
| | B_44:03 |
| | B_44:09 |
| | B_44:27 |
| | B_45:01 |
| | B_83:01 |
| Cluster 9 | B_13:01 |
| | B_13:02 |
| | B_15:01 |
| | B_15:02 |
| | B_15:03 |
| | B_15:08 |
| | B_15:09 |
| | B_15:10 |
| | B_15:11 |
| | B_15:17 |
| | B_15:42 |
| | B_18:01 |
| | B_35:01 |
| | B_35:03 |
| | B_35:07 |
| | B_35:08 |
| | B_38:01 |
| | B_38:02 |
| | B_49:01 |
| | B_50:01 |
| | B_51:01 |
| | B_51:08 |
| | B_52:01 |
| | B_53:01 |
| | B_57:01 |
| | B_57:03 |
| | B_58:01 |
| | B_58:02 |

### Molecular Fingerprints Enable the Modeling of Disease Associations

Patient MHC-I binding repertoire has been implicated in outcomes for several viral and cancer-based diseases. To establish that the predicted binding motifs capture some of these high-level phenotypic relationships, whether distances between MHC-I alleles and genotypes capture known trends in the MHC-I disease associations was assessed. At the individual allele level, it was determined whether MHC-I binding motifs could be used to infer HIV viral control. Past work has suggested that observed MHC-I allele associations with HIV viral control are based on the capacity of individual MHC-I alleles to present structurally relevant regions of HIV proteins. Following this rationale, alleles with similar outcomes are expected to have comparable binding preferences. To test this relationship, alleles with known associations to HIV disease progression were hierarchically clustered based on pairwise binding motif KLD distances (FIGS. 4D and 4E). It was found that once the clusters are defined as alleles that feasibly share a binding motif (i.e., bind similar peptides), the ones with comparable disease outcome are grouped together. This grouping indicates that binding motifs predicted on their electrostatic properties preserve known allelic associations with HIV outcome at the individual MHC-I allele level. To extrapolate beyond alleles with known associations, all alleles that demonstrated a binding motif KLD distance ≤1 to an existing HIV cluster motif were selected, and each of the remaining alleles were assigned to the nearest cluster. This extrapolation resulted in 66% of alleles being assigned (3,827). Clusters were generally compact with the median intra-cluster KLD being 0.2692. Interestingly, while there were marginally more allele clusters associated with HIV progression (11 vs 10), 59.7% of alleles were assigned to clusters with a bias to control HIV disease progression. Despite this observation, the overall average odds ratio, weighted by cluster size and calculated across all assigned alleles, was found to be close to 1 (OR=0.91). In essence, this demonstrated that predicted peptide binding signatures could be used to group peptides with known outcome to HIV.

Evolutionary protein divergence of patient MHC-I genotypes has been strongly associated with outcomes to immune checkpoint inhibitors. However, evolutionary distance is a metric defined solely by analysis of the MHC-I protein sequence. To investigate whether the observed effect could also be due to variance in MHC-I binding motifs, MHC-I genotype distance (arithmetic average of all pairwise binding motif KLDs for a given genotype) was solved for patients who were treated with immune checkpoint inhibitors (ICI) using the predicted motifs from HLA-inception. Patients were stratified by genotype distance with patients in the top quartile being labeled as having high genotype distance while the rest of the patients being labeled as having low genotype distance (FIG. 4F). It was found that patients with a higher level of MHC-I binding motif diversity survived longer when treated with ICIs. Together, this data suggests that patients with more diverse peptide repertoire likely benefit from checkpoint blockade.

### Discussion

In this study, a physics-based inception network is employed to probe the signatures of molecular recognition of the MHC-I protein system, an area traditionally dominated by sequence-based analyses. Capitalizing on these traditional approaches, the present disclosure finds that the inception networks trained on the three-dimensional electrostatic potentials of the MHC-I binding pocket combined with limited binding peptide sequence information could be leveraged to confidently predict MHC-I peptide binding motifs across a range of diverse MHC-I alleles. By using the binding motifs from HLA-Inception, all MHC-I proteins were able to be assigned to an MHC-I supertype. Interestingly, it was found that the heterogeneity of MHC-I binding sub-motifs within a given supertype varied significantly, carrying important implications for the continued use of MHC-I supertypes to design broad peptide-based vaccines. Accordingly, the present disclosure shows that the predicted binding motifs can be utilized to perform pan-allele peptide binding prediction at a high level of precision and speed. Furthermore, the comparison of predicted MHC-I binding motifs was shown to recapitulate known disease associations, namely HIV and immune checkpoint inhibitor response.

There are several profound advantages to approaching the prediction of MHC-I binding motifs, and subsequently peptide ligands, from an electrostatic lens. First, one of the biophysical rules that dictate peptide binding is employed. Sequence-centric MHC-I prediction methods do not explicitly access the underlying forces that drive peptide binding, offering only amino acid configurations that lead to a particular binding motif. This understandably makes such approaches highly biased by variations in binding pocket sequences, which is problematic given the polymorphic nature of the MHC-I protein. In contrast, by training the presently disclosed model directly on the underlying forces, HLA-Inception is able to leam a measurable physical quantity that is ubiquitous to peptide binding and simultaneously tracks with the variations of the pocket sequences and heterogeneity of the motifs. This physics formulation enhances interpretability of binding predictions, which is immediately evident by the results of the leave-one-cluster-out analysis. Another advantage of the shift to electrostatic modeling is a striking reduction of the experimental search space. Because electrostatic potential is a degenerative property, as many different sequence configurations can produce similar local electrostatic environments, the number of MHC-I alleles with unknown binding motifs that require experimental validation is significantly diminished. This makes universal coverage of all human MHC-I binding motifs an experimentally tractable goal, and therefore, opens new doors to broadened applications of T cell-based immunotherapies. Finally, HLA-Inception embodies a methodological advance in computational immunology. The presently disclosed approach is able to perform MHC-I peptide binding prediction without information on non-binding peptides, a common challenge in ML-based peptide binding classification. This precision of predicting motifs and communities, coupled with the ability to infer binding affinities, makes HLA-Inception a natural complement for high throughput MHC-I ligand identification techniques such as mass spectrometry.

There are some caveats to the current implementation stemming from the compositional bias of the training set, the use of nonameric peptide binding motifs, and utilizing a single biophysical descriptor. Like most machine learning models, predictions are biased by the composition of the training set. In cases where the training set provides a good sampling of the total input space, predictions have a high likelihood of accuracy. Conversely, in cases where isolated populations, not captured by the training set, exist then predictions are unlikely to be accurate for these groups. The immense number of MHC-I variants makes this a valid concern for any machine leaming approach to MHC-I ligand prediction and is not specific to the disclosed model. However, as outlined above, we expect that the approach outlined herein will be less affected by this problem due to the learning of the underlying physical nature of peptide binding. To combat this problem, future work can be focused on the experimental resolution of MHC-I alleles with predicted electrostatic environments that fall outside the currently observed distribution. Next, predictions were only done with respect to nonameric peptide binding motifs. This decision was due to the majority of observed peptides being 9 amino acids in length. This translated into high-resolution binding motifs. However, there is a smaller but relevant population of peptides at different lengths. An approach analogous to NN-align was used to extend the nonameric motifs to peptides of lengths 8-11. Reasonably high accuracy for peptides of these lengths was observed, which cover 95% of all observed MHC-I peptides (FIGS. 16C and 16D). FIGS. 16A-16D depict the recall of naturally presented peptides comparing HLA-Inception thresholds at the 0.5% and 2% thresholds as well as the observed Matthew's correlation coefficient for predictions of peptides at different lengths. The observed spearman's rho coefficient between the calculated PWM score and peptide affinity values for peptides at different lengths is also depicted in the plot of FIG. 16D. Finally, HLA-Inception does not implicitly account for other critical physical forces such as van der Waals interactions. It was observed that a majority of MHC-I sequence groups are adequately described by electrostatic interactions, however, a subset of alleles is optimally described using maps of van der Waals potentials (FIG. 15). It is possible that the overall shape of the binding pocket may play a larger role than the innate electrostatic forces for these groups of alleles. Future work will be focused on optimally combining multiple physical descriptors into one model.

In summary, the presently disclosed inception models enable the discovery of biological design principles, the underlying physics of which can extend beyond the system of interest, and predictions of testable phenotypic properties across a broad range of physical conditions. Going forward, the method of leaming the electrostatic environment to perform motif prediction is readily applicable to numerous applications known for high sequence variability, including MHC-II, protein-protein binding, and TCR-MHC binding.

### Methods

MHC-I binding pocket modeling. All the MHC-I sequences used in this disclosure were obtained from the IGMT-HLA database. MHC-I protein sequences were filtered for those described at the complete canonical lengths (HLA-A: 265 amino acids, HLA-B: 362 amino acids, HLA-C: 366 amino acids). This resulted in the consideration of 5,821 sequences from which the peptide binding pocket residues were extracted (residues 25-210).

Modeling of all 5,821 MHC-I alleles began with the selection of templates for homology modeling. 606 potential templates for MHC-I binding pocket modeling were initially identified using the IEDB database and downloaded from the RCSB Protein Data Bank. These 606 structures were then scored using the Molprobity software, an algorithm that ranks a structure based on several stereochemical metrics with lower scores indicating higher quality structures. Of the 606 potential structures, the ones with the lowest Molprobity scores for each unique MHC-I allele were selected, resulting in a total of 50 MHC-I templates. The peptide was removed from each template model, and the protein structure was truncated to only include the peptide binding pocket (reside 25-210 of the amino acid sequence). The templates were then minimized using the default Rosetta score function.

Selected templates were used to model 5,821 MHC-I binding pockets via the following 3-step protocol. First, the peptide pocket (residue 25-210) of each of the 5,821 alleles were aligned by amino acid sequence to all 50 template sequences, and the template structure showing the best alignment (lowest number of necessary mutations) were selected for that allele. Second, computational peptide binding pocket models were generated by mutating each assigned template structure to match the target allele sequence using the Rosetta backrub application with default parameters. Third, an ensemble of 40 structures were generated by selecting the lowest energy models from 40 separate iterations of the Rosetta relax application. Completion of these three steps resulted in a total of 232,840 unique structures (5,281 alleles×40 ensemble members/allele).

Electrostatic map generation. The electrostatic environment of the binding pockets was determined using the APBS software. Each of the 40 ensemble members for a given MHC-I binding pocket were converted into PQR files using the pdb2pqr30 function. The electrostatic potential was then calculated using the default parameters for APBS. The grid dimensions were set to 129 Å×161 Å×129 Å with the fine grid extending to 24 Å beyond the boundaries of the binding pocket and the coarse grid extending to 12 Å beyond the fine grid. Using these parameters, the electrostatic potential was determined at 1 Å resolution, discretizing the three-dimensional space into voxels each containing approximately 1 Å³ of volumetric features. The potentials were calculated using a linearized Poisson-Boltzmann equation with a protein dielectric of 2, a solvent dielectric of 78.54, and an ion concentration of 0.15M. The resulting electrostatic and van der waals maps were then separately saved in the .dx file format. To study the role of charge screening in detecting complementary motifs, the previous set-up was repeated exactly with the exception of the salt concentration being set to 1.5M. The correlation between full electrostatic maps was defined as the element-wise Pearson correlation between two vectorized maps.

MHC-I binding pocket electrostatic map segmentation. The overall process of data generation and training of the HLA Inception Model is depicted in FIGS. 5A-5C. To increase attention on important binding pocket features during inception network training, the electrostatic maps for each MHC-I binding pocket was split into three segments: (1) an N-terminal binding pocket region, (2) a TCR contact region, and (3) C-terminal binding pocket region. Each segment has the dimensions of 12×6×12 voxels with their center coinciding on an evenly spaced vector that runs the length of the binding pocket (FIG. 5A). Features from these segments were employed to predict the amino acid distribution of binding peptide residues that are most likely to reside within these regions. The N-terminal region covered the area that would likely interact with positions 1-3 of binding peptides; the TCR contact region covered the region approximately below positions 4-6 of binding peptide; the C-terminal regions covered the region that would likely interact with positions 7-9 of binding peptides. The segments were then transformed into 3 tensors with each tensor having the dimensions of 5,320×12×6×12. These electrostatic map tensors were then paired a response tensor of the same length containing the amino acid distributions for a single binding peptide residue assigned to that segment. This resulted in 9 training data sets, with a specific training set for each peptide position (FIG. 5B). For example, when training the model to predict the amino acid distribution of position 2 of a binding peptide, the training set would correspond to the tensor of all of the N-terminal segments (5,320×12×6×12) with a response tensor all position 2 amino acid distributions (5,320×20).

### MHC Molecules and Motifs Comparison Metrics

MHC molecule distance metrics. Variation between MHC-I alleles were calculated by employing three metrics, namely Hamming distance, BLOSUM alignment, and Electrostatic Potential Distance, where higher values indicate more divergent alleles. Hamming distance determines the distance between two equal length sequences as the number of mismatches between the two. For example, when comparing the peptides “YMLDLQPET” and “YMLAAQPET”, the number of mismatches (colored in red) are two. Therefore, the hamming distance between the peptides would be two. BLOSUM62 alignment is the sum of the log odds ratios of a particular amino acid substitution given the background frequency of that amino acid. These alignments were calculated with respect to the binding pocket residues within 6 Å of the MHC-I N -and C-terminal anchor residues using the stringDist function in the Biostrings R package. Electrostatic Potential Distance or EPD represents the similarity between electrostatic environments near primary (N- and C-terminal) MHC-I anchor positions. To compute this quantity, a pair of spherical volumes of electrostatic potentials were extracted from the complete binding pocket electrostatic potential environment, one from each terminus. The centers of these spherical volumes were determined using the coordinates of complementary sites on the nonameric peptide ligands. Specifically, after aligning peptides from known MHC-I bound X-ray structures, the centers of the spheres represented the average sidechain centers of mass at peptide positions 2 and 9. A cut-off radius of 6 Å was chosen for defining the volume, as it produces non-overlapping spheres and captures key electrostatic features within one hydration layer. Integrating information from both these anchors, all the n number of three-dimensional voxels embedded within each of the two spherical volumes were concatenated into a single one-dimensional vector. The EPD between any pairwise combinations of such electrostatic vectors (one for each allele) is defined as the Euclidean distance,

$$E P {D}_{xy} = {\sum }_{i = 1}^{n} \sqrt{{( {x}_{i} - {y}_{i} )}^{2}} , \qquad ( 1 )$$

where the i^{th} iso-volumetric voxel occupies the same points in space from two different electrostatic maps corresponding to distinct MHC-I alleles x and y. For the computations in FIG. 2, n=2×4/3 π6³=900 for voxel dimensions of 1 Å³.

MHC peptide binding motif KLD. The similarity of two MHC-I peptide binding motifs, which we call the binding motif KLD, was quantified as the total sum the of observed Kullback-Leibler divergence (KLD) between distributions of amino acids at each position (P1-P9). KLD is a statistical distance measurement that quantifies the divergence of two probability distributions. Kullback-Leibler divergence is calculated using the following equation:

$${D}_{KL} ( P ‖ Q ) = {\sum }_{i = 1}^{k} {P}_{i} log \frac{{P}_{i}}{{Q}_{i}} . \qquad ( 2 )$$

In the above equation, P and Q are discrete probability distributions with k number of bins. Due to the non-symmetry of KLD calculation, i.e., DKL (P∥Q)=/DKL (Q∥P), the KLDs reported in this paper represent a symmetrized KLD computed by finding the average of the KLD values calculated in both directions. The equation is as follows:

$$K L D = \frac{{D}_{KL} ( P ‖ Q ) + {D}_{KL} ( Q ‖ P )}{2} . \qquad ( 3 )$$

The Binding motif KLD was then calculated with the following equation

$$Binding Motif K L D = {\sum }_{i = 1}^{g} K L {D}_{i} . \qquad ( 4 )$$

where i indicates a position of a nonameric peptide and KLDi indicates the observed KLD of the i^{th} position between the two considered MHC-I binding motifs.

K-means clustering of electrostatic potential. K-means clustering of the N- and C-terminal electrostatic potentials at MHC-I anchor positions were performed using the kmeans function from the stats R package. To find the optimal number of clusters, cluster centers were randomly instantiated and added until a cluster containing only a single MHC-I allele was formed. The entire kmeans clustering process, i.e., starting with one cluster and iteratively adding new clusters until termination, was repeated 1000 times with different random seeds and centers were iteratively added until the formation of a single allele cluster was achieved. Following the 1,000 iterations, the optimal number of clusters was determined by finding the most probable termination point across all 1,000 iterations. The overall distribution of termination points across each clustering iteration can be seen in FIG. 7 with the median being 22 clusters.

The terminal potentials were grouped into 22 clusters and were graphically represented using a Voronoi diagram. The center of each Voronoi cell was defined as the average xy-coordinate of each cluster following multidimensional scaling or MDS transformation. In short, the concatenated terminal potential vectors were first transformed into two-dimensional Cartesian space using MDS. The center was then determined by averaging over the reduced MDS coordinates with respect to each cluster. This resulted in a single average xy-coordinate for each cluster, signifying the center of the cluster in the xy-plane.

The compactness of clusters, in terms of the similarity of associated MHC-I binding motifs, was assessed by measuring the intra-cluster and inter-cluster average peptide binding motif KLDs. Intra-cluster average binding motif KLD corresponds to the average of binding motif KLDs of all pairwise combinations of alleles within a cluster. The inter-cluster average binding motif KLD represents the average binding motif KLD of all pairwise of combinations of alleles within a cluster to all alleles not contained within the cluster. A large difference between intra-cluster and inter-cluster average KLDs indicates a compact and non-redundant group.

### HLA Inception Model

Training set of MHC-I peptide binding motifs. The training set of MHC-I binding motifs were determined using MHC-I peptides with experimentally known binding affinities. The peptide data was filtered to select for only binding peptides that were nine amino acids in length and were assigned to an allele at four-digit resolution. To ensure these binding motifs have comprehensive amino acid representation at each position, only alleles with at least 50 experimentally validated binders were selected. Following the constraints of peptide length, resolution, and binding affinity, we could train the neural networks on 133 out of 5,821 MHC-I alleles. For each of these 133 alleles, all the 50 or more assigned peptides were then aligned, and the amino frequency at each position was calculated. Finally, these position-specific amino frequencies were used to define the peptide binding motif for a given MHC-I allele.

Model architecture and training. HLA-Inception is modeled after the inception v1 developed by google®. The architecture of HLA-Inception consists of one inception module followed by four densely connected layers that are separated by dropout layers. The output layer returns a one-dimensional vector of length 20 with the loss being calculated using a KLD loss function and optimized using the ADAM algorithm for stochastic gradient dissent. Overall, HLA-Inception consists of a collection of nine individual models, each corresponding to a different position of the peptide binding motif. A hyperparameter search was performed to identify the best number of epochs and learning rate. Due to the general importance of position 2 and position 9, a grid search was performed on these positions covering epochs 50, 75, and 100 and learning rates 1e-2, 1e-3, and 1e-4. 100 epochs and a leaming rate of 1e-3 were identified as the most optimal and were used to train all 9 models when performing 10-fold cross-validation (FIGS. 8A-8T).

Motif prediction. Using the optimal parameters, HLA-Inception was trained on available structure and binding data from 133 alleles and used to predict binding motifs for across all 5,821 alleles. In order to provide enhanced context for predictions, maps corresponding to each of the 40-structure ensembles were averaged to produce allele-specific potential representations. The averaged maps were then segmented, as previously described, and used as inputs to the trained model for predicting their associated binding motifs. Full binding motifs were then generated by combining the predictions from all nine HLA-Inception models (FIGS. 5A-B).

### MHC-I Supertype Analysis

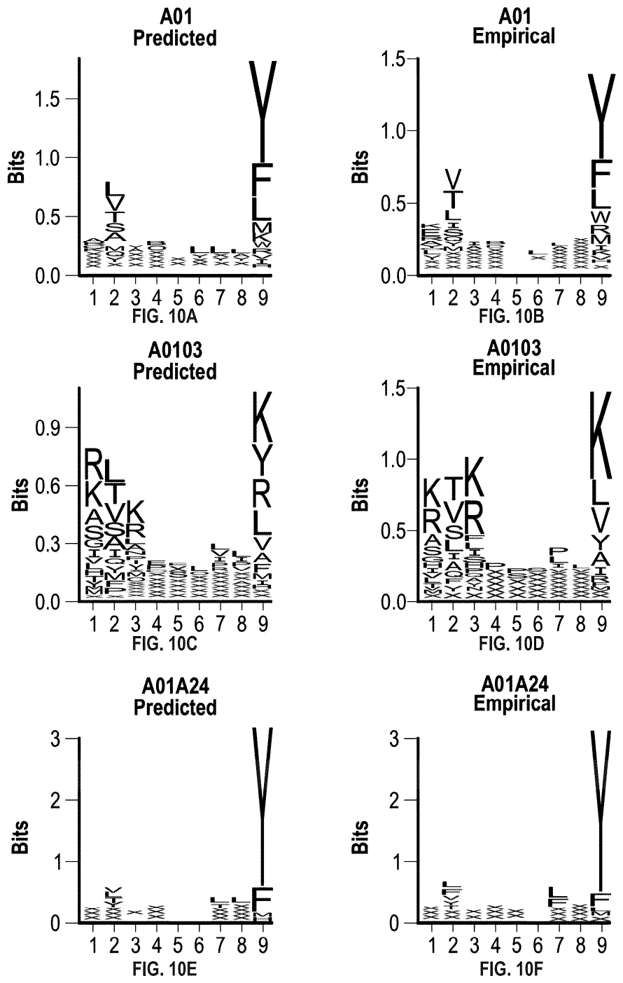

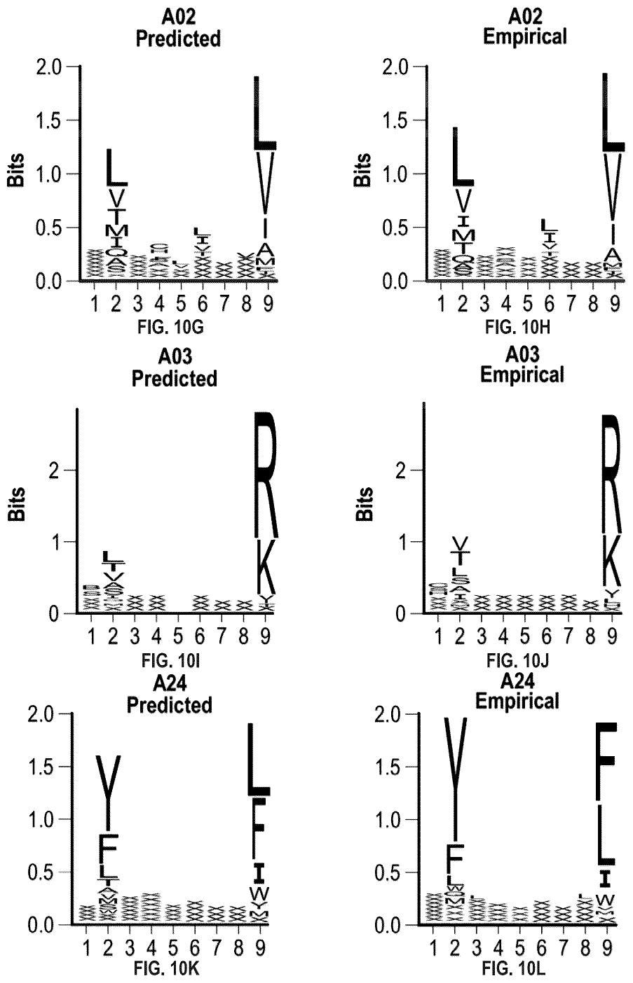

MHC-I supertypes. Classical MHC-I supertypes were defined as alleles described as reference panel alleles in Sidney et al. Using the defined alleles supertype definitions, the predicted binding motifs determined from HLA-Inception or the experimental binding motifs determined from the peptide data were averaged together to represent each supertype and plotted in FIGS. 10A-12L. However, the classical supertypes failed to incorporate most HLA-C alleles. To avoid a large number of unmapped MHC-I binding motifs, six new HLA-C allele-specific supertypes were generated, namely C01, C02, C03, C04, C06, C07. The new HLA-C supertypes were assigned by inspecting logo plots for all HLA-C alleles with at least 100 known binders, and grouping alleles with visually similar plots. A table of reference alleles for each HLA supertype can be seen in FIG. 14 and representative logo plots for each supertype can be seen in FIG. 10.

MHC-I supertype graph. A graphical representation describing the topology of the MHC-I supertype network was initiated using an unconnected network, where each node was defined as a different MHC-I supertype binding motif. These MHC-I supertype binding motifs were created by averaging the predicted binding motifs for a set of reference alleles assigned to a given supertype (Table 3, FIGS. 10A-12L). The connections or edges between the nodes were then added by measuring the pairwise binding motif KLDs (eq. 4) between all supertypes binding motifs; longer edges imply higher inter-motif divergence. In order to generate a more informative graph, where similar motifs would spatially cluster together, the edges of the fully connected network were trimmed. This trimming procedure consisted of connecting all nodes to the top three most similar nodes (not including itself) as determined by minimizing binding motif KLD. Extreme edges (binding motif KLD≥4) were also removed. It is important to note that some nodes will have more than three connections if that node is within the top-three lowest binding motif KLDs of multiple supertypes. The trimmed network was then visualized using the Kamada-Kawai force-directed algorithm. Following the generation of the MHC-I supertype graph, binding motif KLDs were remeasured for pairwise combinations between supertype binding motifs and HLA-inception-predicted individual allele binding motifs. Each individual binding motif was then assigned to the supertype that produced the lowest binding motif KLD.

TABLE 3
Supertype Assignment

| | |
|---|---|
| A01 | A*01:01 |
| | A*26:01 |
| | A*26:02 |
| | A*26:03 |
| | A*30:02 |
| | A*30:03 |
| | A*30:04 |
| | A*32:01 |
| A0103 | A*30:01 |
| A01A24 | A*29:02 |
| A02 | A*02:01 |
| | A*02:02 |
| | A*02:03 |
| | A*02:04 |
| | A*02:05 |
| | A*02:06 |
| | A*02:07 |
| | A*02:14 |
| | A*02:17 |
| | A*68:02 |
| | A*69:01 |
| A03 | A*03:01 |
| | A*11:01 |
| | A*31:01 |
| | A*33:01 |
| | A*33:03 |
| | A*66:01 |
| | A*68:01 |
| | A*74:01 |
| A24 | A*23:01 |
| | A*24:02 |
| B07 | B*07:02 |
| | B*07:03 |
| | B*07:05 |
| | B*15:08 |
| | B*35:01 |
| | B*35:03 |
| | B*42:02 |
| | B*51:01 |
| | B*51:02 |
| | B*53:01 |
| | B*54:01 |
| | B*55:01 |
| | B*55:02 |
| | B*56:01 |
| | B*67:01 |
| | B*78:01 |
| B08 | B*08:01 |
| | B*08:02 |
| B27 | B*14:02 |
| | B*15:03 |
| | B*15:09 |
| | B*15:10 |
| | B*15:18 |
| | B*27:02 |
| | B*27:03 |
| | B*27:04 |
| | B*27:05 |
| | B*27:06 |
| | B*27:07 |
| | B*27:09 |
| | B*38:01 |
| | B*39:01 |
| | B*39:09 |
| | B*48:01 |
| | B*73:01 |
| B44 | B*18:01 |
| | B*37:01 |
| | B*40:01 |
| | B*40:02 |
| | B*40:06 |
| | B*44:02 |
| | B*44:03 |
| | B*45:01 |
| B58 | B*15:16 |
| | B*15:17 |
| | B*57:01 |
| | B*57:02 |
| | B*58:01 |
| | B*58:02 |
| B62 | B*15:01 |
| | B*15:02 |
| | B*15:12 |
| | B*15:13 |
| | B*46:01 |
| | B*52:01 |
| C01 | C*01:02 |
| C02 | C*02:02 |
| | C*03:02 |
| | C*12:02 |
| | C*12:03 |
| | C*16:01 |
| C03 | C*03:03 |
| | C*03:04 |
| | C*08:01 |
| | C*15:02 |
| C04 | C*04:01 |
| | C*04:03 |
| | C*05:01 |
| | C*08:02 |
| C06 | C*06:02 |
| | C*07:01 |
| | C*07:02 |
| C07 | C*07:04 |
| | C*14:02 |
| | C*14:03 |

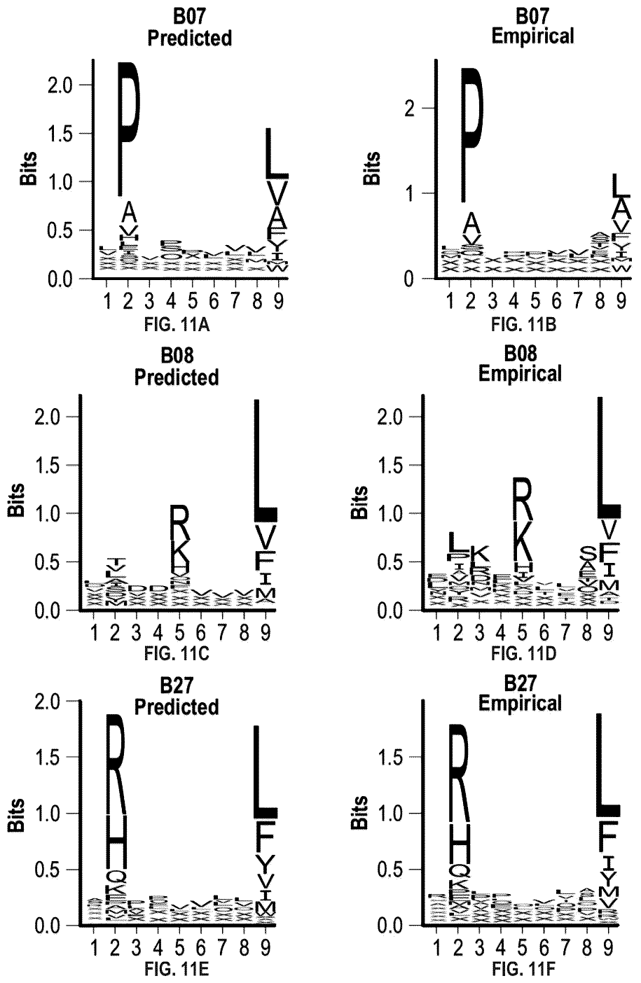

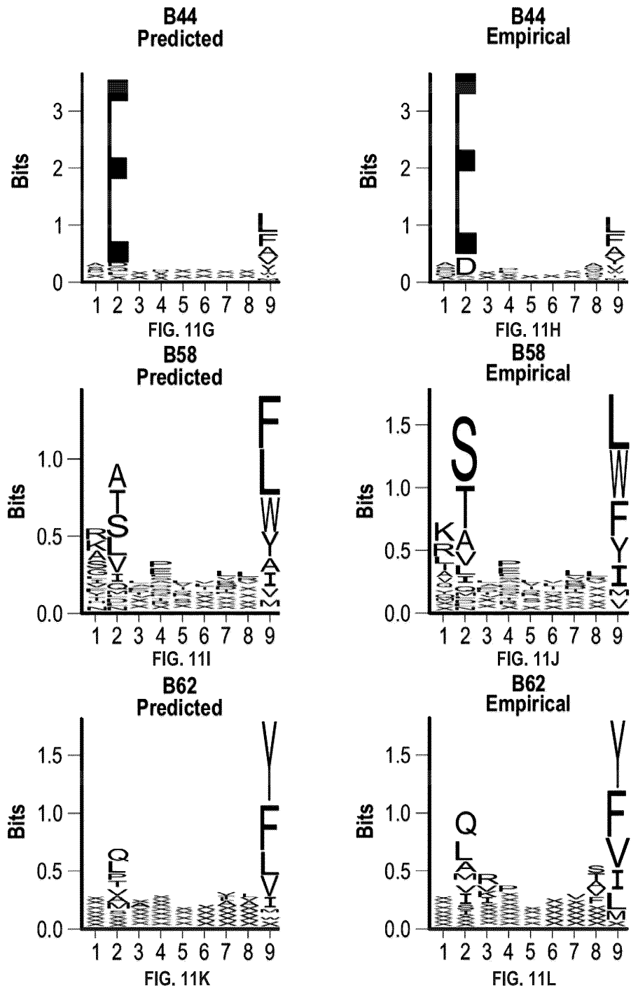

Supertype subgraph generation and analysis. Subgraphs consisting of all the MHC-I alleles assigned to a particular supertype were generated (FIGS. 3E-3H, 11E). In these supertype-specific subgraphs, each node represented a different individual allele assigned to that supertype, and the edges between alleles was defined as the binding motif KLD between alleles. Similar to the supertype graph, edges between nodes in the subgraph were trimmed. In this case, edges were trimmed to remove any connection between alleles that are unlikely to bind the same peptides (Binding motif KLD≥1). For more information about the selection of the KLD thresholds, see the next section. Peptide binding sub-motifs and heterogeneity (modularity) within each supertype subgraph were detected and calculated using the infomap algorithm.

Cross-allele binding predictions. The relationship between binding motif KLD and quantitative changes in binding affinity was assessed by performing cross-allele binding predictions (FIGS. 13B-C). Cross-allele binding predictions involve the process of taking peptides with known affinity to one allele and predicting the affinity of those peptides to a different allele. In this study, cross-allele binding predictions were used in two contexts: Assessing the impact of MHC-I supertype heterogeneity and determining a binding motif KLD threshold for shared binding.

In the context of MHC-I supertype heterogeneity, two different MHC-I alleles classically assigned to a given supertype (e.g., A02 or B44) were selected, and the binding motif KLD between those two alleles were calculated using their predicted motifs from HLA-inception. Next, 1,000 peptides with experimentally known binding affinities were randomly sampled from each of the two alleles to create a peptide set of 2,000 total peptides. NetMHCpan-4.1 was then used to predict the affinity of all peptides experimentally validated to bind to one allele on the other allele and vice versa. The average binding affinity was calculated with respect to all NetMHCpan-4.1 predictions, representing the expected cross-allele binding affinity. To determine the relative change in expected binding affinity, the calculated cross-allele affinity values were subtracted from the average observed affinity of peptide ligands assigned to that allele (FIG. 13C).

A binding motif KLD threshold where alleles with that value or lower are expected to bind similar peptides is important for grouping alleles and estimating the potential clinical associations. To find this binding motif KLD threshold, a linear model was fit to all allele pairs with a binding motif KLD of less than 1.5 and compared to the average cross-allele binding predictions (FIG. 13B). A binding motif KLD of 1 was shown to indicate an average affinity of 5,000 nM, and was selected as the binding motif KLD threshold.

### MHC-I Ligand Prediction

Sequence-based model. A deep sequence-based model, inspired by Nielsen et al, was constructed for comparison to HLA-Inception (FIG. 15). The input to this model was a BLOSUM-encoded vector of key positions within the MHC-I binding pocket, and the model was trained on a balanced data set consisting of 315,512 experimentally resolved MHC-I peptides paired with randomly generated decoy peptides. The model consisted of 3 densely connected layers separated by dropouts, offering a comparable architecture to HLA-Inception. The output of the model was the probability of the given peptide being a binder.

Position-weighted matrix score. Position-weighted matrix (PWM) score is a measure of how strongly a peptide adheres to the probability distribution underlying a given binding motif. The PWM score is calculated by the sum of the log-odd ratios of observing an amino acid at a particular position, given the background frequency of that amino acid within the motif. The equation to calculate PWM is as follows,

$$P W M score ( pep ) = {\sum }_{i = 1}^{g} log \frac{{p}_{ij}}{{q}_{j}} . \qquad ( 5 )$$

where pep is a peptide being scored, i is the residue number being considered, pᵢⱼ is the probability of the i-th residue of pep at the i-th position according to the binding motif, and qⱼ is the background frequency of the i-th residue of pep. A higher PWM score indicates a higher probability of a peptide binding to a target allele. Allele-specific score thresholds were determined by calculating the PWM scores for all nonameric peptides in the human protein and generating a cdf of that distribution.

Correlation with quantitative peptide binding scores. Peptides with quantitative values for binding affinity, IC50 or complex stability, were extracted from the IEDB database. Peptide were selected according to the same criteria as peptide selection for the generation of experimental peptide binding motifs. Correlations between quantitative binding values and PWM score were calculated using spearman correlation.

Presentation prediction of natural ligands. For analysis centered on the recovery of naturally presented MHC-I ligands, 9mer MHC-I peptides with experimental evidence of presentation for 50 different alleles were obtained from the HLA Ligand Atlas. For each allele, 1,000 peptides experimentally determined to be naturally presented were randomly sampled and combined with 99,000 decoy peptides (not naturally presented) extracted from the human proteome. In cases where alleles had less than 1,000 experimentally described peptides, all peptides were used and decoys peptides were sampled to maintain a ratio of 1:100 target-decoy ratio.

Leave one cluster out analysis. Leave one cluster out analysis is defined as the process of using a cluster of alleles, defined by similar binding pocket sequences, to test the generalizability of different models on unseen data. MHC-I sequences were clustered with respect to BLOSUM-encoded key positions from the MHC-I binding pocket (described in Nielsen et al.). The optimal number of clusters, 11, was determined using the average silhouette width method implemented in the fviz_nbclust function in the factoextra R package. Following clustering, models would then be tested on each identified cluster by withholding all alleles assigned to a given cluster from the training set, and then testing model performance on those withheld alleles. The leave one cluster out analysis was performed on all clusters. Model accuracy was reported as the individual Matthew's correlation coefficients for peptide prediction of each allele within the cluster. The leave one out cluster analysis was also performed for HLA-Inception models trained on high salt concentration electrostatic maps and van der Waals maps (FIG. 15).

Proteome-scale peptide predictions. Full Human (UP000005640) and Mouse (UP000000589) proteomes were extracted from the uniprot database, and all reference protein sequences for viruses (Virus) with homo sapiens listed as a host was extracted from the ncbi virus database. All predictions were performed using an apple M1 pro chip with 10 cpu cores.

### Individual Allele and Genotype Disease Associates

Hierarchical clustering of alleles associated with HIV outcome. MHC-I alleles showing statistically significant associations with HIV disease progression were hierarchically clustered based on pairwise binding motif KLD calculations using the WPGMA method. Clusters of alleles were identified by cutting the tree at a KLD threshold of 1. The overall log odds ratio of a cluster was defined as the average of individual log odds ratios of alleles contained within a cluster. Predicted binding motifs were then assigned to an HIV outcome associated allele cluster by calculating the distance between a given allele to all cluster motifs and assigning the allele based on minimal binding motif KLD distance.

Analysis of immune checkpoint data. A dataset describing survival following immune checkpoint blockade of 314 melanoma and non-small cell lung cancer patients was obtained from Chowell et al. Each patient in the cohort was assigned a genotype distance value that corresponded to the average of all pairwise binding motif KLDs for that patents' MHC-I genotype. Patients with genotype distances in the top 75% were labeled as having high genotype distance while all other patients were labeled as having low genotype distance. A Kaplan-Meier plot describing the survival rates of both groups was generated using ggsurvfit. A hazard ratio between the two groups was determined by fitting a cox proportional hazards regression model to the data.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

### Further Example Implementations and Features

In some aspects, the systems and methods disclosed herein may be implemented in the development of vaccines. In cases where the need for a vaccine exists for a population, a specific MHC-I molecule that is highly expressed in the population can be selected for prediction of the amino acid sequence of its corresponding binding motif peptide. The predicted amino acid sequence of the binding motif peptide can then be used to design a peptide antigen for a vaccine that will be suitable for the population.

In another possible implementation, the disclosed systems and methods can be used to determine whether a subject is a possible candidate for immune checkpoint inhibitor therapy. First, the MHC-I binding motif prediction method can be used to predict the amino acid sequence of the binding motif peptides of a plurality of MHC-I alleles expressed by the subject. Using the diversity of the predicted amino acid sequences the diversity of the MHC-I alleles can be quantified. By comparing the diversity of the MHC-I alleles in the subject to a predetermined threshold, it can be determined whether the subject is a candidate for immune checkpoint inhibitor therapy if the diversity of the MHC-I alleles is higher than the predetermined threshold.

In yet another implementation, the disclosed systems and methods can be used to determine a risk of HIV disease progression in a subject. The MHC-I binding motif prediction method can be used to predict the amino acid sequence of the binding motif peptides of one or more MHC-I alleles expressed by the subject. The amino acid sequence of each predicted binding motif peptide can then be compared to an amino acid sequence of one or more HIV-associated proteins. This comparison can thus be used to determine the risk of HIV-disease progression in the subject based on the similarity or sequence identity between the predicted amino acid sequence(s) of the one or more MHC-I alleles and the amino acid sequence(s) of the one or more HIV-associated proteins.

In some embodiments, the disclosed amino acid sequence prediction methods and systems can be used to predict either the amino acid sequence of an MHC-I binding motif peptide or the amino acid sequence of a peptide binding to an MHC-I. As such, it should be understood from the present disclosure that the same methods and systems described for the prediction of the amino acid sequence of an MHC-I binding motif peptide are used when predicting the amino acid sequence of a peptide binding to an MCH-I. A method for predicting an amino acid sequence of a binding peptide to an MHC-I can therefore include modeling a binding pocket of the MHC-I which is binding to the peptide. The model of the binding pocket of the MHC-I can be performed by selecting a template model that has the most similar amino acid sequence to that of the MHC-I from a plurality of existing MHC-I binding pocket template models, generating an ensemble of binding pocket structures by mutating the selected template model to match the amino acid sequence of the MHC-I, and averaging the ensemble of binding pocket structures to obtain an average binding pocket model.

After modeling the binding pocket of the MHC-I, an electrostatic potential distribution of the model of the binding pocket may be generated. The electrostatic potential distribution can be a three-dimensional electrostatic potential grid of the binding pocket of the MHC-I and can be divided into three volumes that each correspond to a separate region of the binding peptide. These regions may correspond to the N-terminal binding pocket, the TCR contact region, and the C-terminal binding pocket. A ML model can then be executed using biophysical data derived from the electrostatic potential distribution generated from the model of the binding pocket to predict the amino acid sequence of the peptide binding to the MHC-I. In some embodiments, the ML model can apply the three volumes of the electrostatic potential distribution to predict a distinct portion of the amino acid sequence of the binding motif peptide. In yet further embodiments the three volumes of the electrostatic potential distribution are passed through the ML model using an output layer of amino acid distributions at the respective position of each amino acid in a peptide binding to an MHC-I binding motif. The ML model can thus predict the amino acid sequence of the peptide binding to the MHC-I once the ML model has converged for all respective positions of each amino acid in the binding peptide.

What is claimed is:

1. A method for predicting an amino acid sequence of a binding motif peptide of a Major Histocompatibility Complex I (MHC-I), the method comprising:
(a) modeling a binding pocket of the MHC-I;
(b) generating an electrostatic potential distribution of the model of the binding pocket;
(c) executing a machine learning (ML) model using biophysical data derived from the electrostatic potential distribution of the model of the binding pocket of the MHC-I; and
(d) predicting the amino acid sequence of the binding motif peptide of the MHC-I based on the biophysical data.

2. The method of claim 1, wherein the electrostatic potential distribution comprises a three-dimensional electrostatic potential grid of the binding pocket of the MHC-I.

3. The method of claim 1, wherein the electrostatic potential distribution comprises three volumes corresponding to an N-terminal binding pocket, a TCR contact region, and a C-terminal binding pocket.

4. The method of claim 1, wherein the electrostatic potential distribution comprises three volumes corresponding to an N-terminal binding pocket, a TCR contact region, and a C-terminal binding pocket which are applied by the ML model to predict a distinct portion of the amino acid sequence of the binding motif peptide.

5. The method of claim 1, wherein the ML model is a deep convolutional neural network that models diversity of MHC-I peptide binding pockets and is trained using electrostatic potential distributions corresponding to amino acid distribution of MHC-I peptide binding pockets and experimentally verified binding motif peptides.

6. The method of claim 1, wherein the predicted amino acid sequence is 8 to 12amino acids in length.

7. The method of claim 1, wherein the predicted amino acid sequence is 9 amino acids in length.

8. The method of claim 1, wherein the MHC-I has an unknown, unpredicted, or otherwise not experimentally verified, peptide interaction signature.

9. The method of claim 1, wherein modeling the binding pocket of the MHC-I comprises:
selecting a template model from a plurality of existing MHC-I binding pocket template models having an amino acid sequence most similar to an amino acid sequence of the MHC-I;
generating an ensemble of binding pocket structures by mutating the selected template model to match the amino acid sequence of the MHC-I; and
averaging the ensemble of binding pocket structures to obtain an average binding pocket model.

10. A computer-implemented system for prediction of MHC-I binding motif peptides, comprising:
a processor; and
a memory comprising machine-executable instructions in operable communication with the processor, the instructions executable by the processor to:
access biophysical measurements of an MHC-I binding pocket, wherein the biophysical measurements comprise structural data associated with the MHC-I binding pocket;
generate an electrostatic potential distribution based on the structural data associated with the MHC-I binding pocket; and
execute a machine learning (ML) model to predict an amino acid sequence of the MHC-I binding motif peptide using the biophysical measurements of the MHC-I binding pocket and the electrostatic potential distribution.

11. The system of claim 10, wherein the ML model is a deep convolutional neural network that models diversity of MHC-I binding pockets and is trained using electrostatic potential distribution corresponding to amino acid distribution of MHC-I binding pockets and experimentally verified binding motif peptides.

12. The system of claim 10, wherein the ML model is trained on three dimensional representations of the electrostatic potential within the MHC-I binding pocket to reproduce sequences of known binding motif peptides such that following training, the ML model can predict binding motif peptide sequences for MHC-I alleles with unknown binding motif peptides.

13. The system of claim 10, wherein the biophysical measurements of the MHC-I binding pocket further comprise a structural model of the MHC-I binding pocket.

14. The system of claim 10, wherein the electrostatic potential distribution is subdivided into three volumes corresponding to an N-terminal binding pocket, a TCR contact region, and a C-terminal binding pocket of the MHC-I binding pocket.

15. The system of claim 10, wherein the system predicts MHC-I binding motif peptides that are 8-12 amino acids in length.

16. The system of claim 10, wherein the system predicts MHC-I binding motif peptides that are 9 amino acids in length.

17. A method for predicting an amino acid sequence of a binding peptide to a Major Histocompatibility Complex I (MHC-I), the method comprising:
(a) modeling a binding pocket of the MHC-I;
(b) generating an electrostatic potential distribution of the model of the binding pocket;
(c) executing a machine learning (ML) model using biophysical data derived from the electrostatic potential distribution of the model of the binding pocket of the MHC-I; and
(d) predicting the amino acid sequence of the binding peptide to the MHC-I based on the biophysical data.

18. The method of claim 17, wherein the electrostatic potential distribution comprises three volumes corresponding to an N-terminal binding pocket, a TCR contact region, and a C-terminal binding pocket which are applied by the ML model to predict a distinct portion of the amino acid sequence of the binding peptide to the MHC-I.

19. The method of claim 17, wherein the ML model is a deep convolutional neural network that models diversity of MHC-I peptide binding pockets and is trained using electrostatic potential distributions corresponding to amino acid distribution of MHC-I peptide binding pockets and experimentally verified binding motif peptides.

20. The method of claim 17, wherein modeling the binding pocket of the MHC-I comprises:
selecting a template model from a plurality of existing MHC-I binding pocket template models having an amino acid sequence most similar to an amino acid sequence of the MHC-I;
generating an ensemble of binding pocket structures by mutating the selected template model to match the amino acid sequence of the MHC-I; and
averaging the ensemble of binding pocket structures to obtain an average binding pocket model.
